SEQUENCE LISTING (1 sequence)

SEQ ID NO: 1 - DNA, 41902 bp, Homo sapiens
  source 1..41902: mol_type = genomic DNA
ctctcctcgcggcgcgagtttcaggcagcgctgcgtcctgctgcgcacgtgggaagccctggccccggccacccccgcgatgccgcgcgctccccgctgccgagccgtgcgctccctgctgcgcagccactaccgcgaggtgctgccgctggccacgttcgtgcggcgcctggggccccagggctggcggctggtgcagcgcggggacccggcggctttccgcgcgctggtggcccagtgcctggtgtgcgtgccctgggacgcacggccgccccccgccgccccctccttccgccaggtgggcctccccggggtcggcgtccggctggggttgagggcggccggggggaaccagcgacatgcggagagcagcgcaggcgactcagggcgcttcccccgcaggtgtcctgcctgaaggagctggtggcccgagtgctgcagaggctgtgcgagcgcggcgcgaagaacgtgctggccttcggcttcgcgctgctggacggggcccgcgggggcccccccgaggccttcaccaccagcgtgcgcagctacctgcccaacacggtgaccgacgcactgcgggggagcggggcgtgggggctgctgctgcgccgcgtgggcgacgacgtgctggttcacctgctggcacgctgcgcgctctttgtgctggtggctcccagctgcgcctaccaggtgtgcgggccgccgctgtaccagctcggcgctgccactcaggcccggcccccgccacacgctagtggaccccgaaggcgtctgggatgcgaacgggcctggaaccatagcgtcagggaggccggggtccccctgggcctgccagccccgggtgcgaggaggcgcgggggcagtgccagccgaagtctgccgttgcccaagaggcccaggcgtggcgctgcccctgagccggagcggacgcccgttgggcaggggtcctgggcccacccgggcaggacgcgtggaccgagtgaccgtggtttctgtgtggtgtcacctgccagacccgccgaagaagccacctctttggagggtgcgctctctggcacgcgccactcccacccatccgtgggccgccagcaccacgcgggccccccatccacatcgcggccaccacgtccctgggacacgccttgtcccccggtgtacgccgagaccaagcacttcctctactcctcaggcgacaaggagcagctgcggccctccttcctactcagctctctgaggcccagcctgactggcgctcggaggctcgtggagaccatctttctgggttccaggccctggatgccagggactccccgcaggttgccccgcctgccccagcgctactggcaaatgcggcccctgtttctggagctgcttgggaaccacgcgcagtgcccctacggggtgctcctcaagacgcactgcccgctgcgagctgcggtcaccccagcagccggtgtctgtgcccgggagaagccccagggctctgtggcggcccccgaggaggaggacacagacccccgtcgcctggtgcagctgctccgccagcacagcagcccctggcaggtgtacggcttcgtgcgggcctgcctgcgccggctggtgcccccaggcctctggggctccaggcacaacgaacgccgcttcctcaggaacaccaagaagttcatctccctggggaagcatgccaagctctcgctgcaggagctgacgtggaagatgagcgtgcgggactgcgcttggctgcgcaggagcccaggtgaggaggtggtggccgtcgagggcccaggccccagagctgaatgcagtaggggctcagaaaagggggcaggcagagccctggtcctcctgtctccatcgtcacgtgggcacacgtggcttttcgctcaggacgtcgagtggacacggtgatctctgcctctgctctccctcctgtccagtttgcataaacttacgaggttcaccttcacgttttgatggacacgcggtttccaggcgccgaggccagagcagtgaacagaggaggctgggcgcggcagtggagccgggttgccggcaatggggagaagtgtctggaagcacagacgctctggcgagggtgcctgcaggttacctataatcctcttcgcaatttcaagggtgggaatgagaggtggggacgagaaccccctcttcctgggggtgggaggtaagggttttgcaggtgcacgtggtcagccaatatgcaggtttgtgtttaagatttaattgtgtgttgacggccaggtgcggtggctcacgccggtaatcccagcactttgggaagctgaggcaggtggatcacctgaggtcaggagtttgagaccagcctgaccaacatggtgaaaccctatctgtactaaaaatacaaaaattagctgggcatggtggtgtgtgcctgtaatcccagctacttgggaggctgaggcaggagaatcacttgaacccaggaggcggaggctgcagtgagctgagattgtgccattgtactccagcctgggcgacaagagtgaaactctgtctttaaaaaaaaaaagtgttcgttgattgtgccaggacagggtagagggagggagataagactgttctccagcacagatcctggtcccatctttaggtatgaagagggccacatgggagcagaggacagcagatggctccacctgctgaggaagggacagtgtttgtgggtgttcaggggatggtgctgctgggccctgccgtgtccccaccctgtttttctggatttgatgttgaggaacctccgctccagcccccttttggctcccagtgctcccaggccctaccgtggcagctagaagaagtcccgatttcaccccctccccacaaactcccaagacatgtaagacttccggccatgcagacaaggagggtgaccttcttggggctcttttttttctttttttctttttatggtggcaaaagtcatataacatgagattggcactcctaacaccgttttctgtgtacagtgcagaattgctaactcggcggtgtttacagcaggttgcttgaaatgctgcgtcttgcgtgactggaagtccctacccatcgaacggcagctgcctcacacctgctgcggctcaggtggaccacgccgagtcagataagcgtcatgcaacccagttttgctttttgtgctccagcttccttcgttgaggagagtttgagttctctgatcaggactctgcctgtcattgctgttctctgacttcagatgaggtcacaatctgcccctggcttatgcagggagtgaggcgtggtccccgggtgtccctgtcacgtgcagggtgagtgaggcgttgcccccaggtgtccctgtcacgtgtagggtgagtgaggcgcggcccccgggtgtccctgtcccgtgcagcgtgattgaggtgtggcccccgggtgtccctgtcccgtgcagggtgagtgaggcactgtccccgggtgtccctgtcacgtgcagggtgagtgaggcgcggtccccgggtgtccctgtcacgtgtagggtgagtgaggcactgtccccgggtgtccctctcaggtgtagggtgagtgaggcgccgtccccgggtgtccctgtcacgtgtagggtgagtgaggcgtggtccccgggtgtccctgtcccgtgcagggtgagtgaggcgctgtccccgggtgtccctgtcacgtgcagggtgagtgaggcgcggtccccgggtgtccctgtcacgtgtagggtgagtgaggcaccgtccctgggtgtccctcccaggtgtagggtgagtgaggcgctgtccccgggtgtccctctcaggtgtagggtgagtgaggcgcggccccaggtgtccctgtcacgtgtagggtgagtgaggcaccgtccctgggtgtccctcccaggtatagggtgagtgaggcactgtccccgggtgtccctgtcacgtgcagggtgagtgaggcgcggtccccaggtgtccctgtcacgtgtagggtgagtgaggcactgtccccgggtgtccctgtcccgtgcagggtgagtgaggcgcggtccccaggtgtccctgtcccgtgcagggtgagtgaggcgccgtccccgggtgtccctgtcacgtgcagggtgagtgaggcacggcccccgggtgtccctgtcacgtgcagggtgagtgaggcgcggcccccgggtgtccctgtcacgtgcagggtgagtgaggcgcggcccccgggtgtccctgtcacgtgcagggtgagtgaggcgcggtccccgggtgtccctgtcacgtgcagggtgagtgaggcgcggtccccgggtgtccctgtcacgtgcagggtgagtgaggcgcggtccccgggtgtccctgtcacgtgcagggtgagtgaggcacggtccccgggtgtccctgtcccgtgtagggtgagtgaggcactgtccccgggtgtccctgtcacgttcagggtgagtgaggcgcggtccccgggtgtccctgtcacgtgtagggtgagtgaggcaccgtccccgggtgtccctgtcacgtgcagggtgagtgaggcgctgtccccgggtgtccctgtcacgtgcagggtgattgacgcgaggcccccgggtgtccctgtcacgtgcagggtgagtgaggcgccgtccccgcgtgtccctgtcacgtgcagggtgagtgaggcgccgtccccgggtgtccctgtcacgtgtagggtgagtgaggtgccgtccccgggtgtccctgtcacgtgtagggtgagtgaggcgccgtccccgggtgtccctgtcacgttcagggtgagtgaggcacggcccccgggtgtccctgtcacgtgtagggtgagtgaggcgcggtccccgggtgtcactgtcacgtgtagggtgagtgaggcactgtccccgggtgtccctgtcacgtgtagggtgagtgaggcgcggcccccgggtgtccctgtcacgtgcagggtgagtgaggcgcggcccccgggtgtccctgtcacgtgtagggtgagtgaggcgcggcccccgggtgtccctgtcacgtgcagggtgagtgaggcgcggcccccgggtgtccctgtcacgtgcagggtgagtgaggcgcggcccccgggtgtccctgtcacgtgcagggtgagtgaggcacggcccccgggtgtccctgtcacgtgcagggtgagtgaggcgcggcccccgggtgtccctgtcacttgcagggtgagtgaggcactgtccccgggtgtccctgtcacgttcagggtgagtgaggcgcggtccccgggtgtccctgtcacgtgtagggtgaatgaggcactgtccccgggtgtccctgtcacgtgcagggtgagtgaggcgccgtccccgggtgtccctgtcacgtgcagggtgattgacgcgaggcccccgggtgtccctgtcacgtgcagggtgagtgaggcgccgtccccgcgtgtccctgtcacgtgcagggtgagtgaggcgccgtccccgggtgtccctgtcacgtgtagggtgagtgaggcgccgtccccgggtgtccctgtcacgtgtagggtgagtgaggcgccgtccccgggtgtccctgtcacgtgtagggtgagtgaggcgccgtccccgggtgtccctgtcacgtgtagggtgagtgaggcgccgtccccgggtgtccctgtcacgtgcagggtgagtgaggccccgtccccgggtgtccctgtcacgtgtagggtgagtgaggcactgtccccgggtgtctctgtcacgtgtagggtgagtgaggcgccgtccctgggtgtccctctcaggtgtagggtgagtgaggcgccatccccgggtgtccctgtcacgtgtagggtgagtgaggcgccgtccccgggtgtccctgtcacgtgtagggtgagtgagttgcggcccccgggtgtccctctcaggtgcagggtgagtgaggcgctgtccctgggtgtccctgtctcgtgtagggtgagtgaggctctgtccccaggtgtccttggcgtttgctcacttgagcttgctcctgaatgtttgctctttctatagccacagctgcgccggttgcccattgcctgggtagatggtgcaggcgcagtgctggtccccaagcctatcttttctgatgctcggctcttcttggtcacctctccgttccattttgctacggggacacgggactgcaggctctcgcctcccgcgtgccaggcactgcagccacagcttcaggtccgcttgcctctgttgggcctggcttgctcaccacgtgcccgccacatgcatgctgccaatactcctctcccagcttgtctcatgccgaggctggactctgggctgcctgtgtctgctgccacgtgttgctggagacatcccagaaagggttctctgtgccctgaaggaaagcaagtcaccccagccccctcacttgtcctgttttctcccaagctgcccctctgcttggcccccttgggtgggtggcaacgcttgtcaccttattctgggcacctgccgctcattgcttaggctgggctctgcctccagtcgccccctcacatggattgacgtccagccacaggttggagtgtctctgtctgtctcctgctctgagacccacgtggagggccggtgtctccgccagccttcgtcagacttccctcttgggtcttagttttgaatttcactgatttacctctgacgtttctatctctccattgtatgctttttcttggtttattctttcattccttttctagcttcttagtttagtcatgcctttccctctaagtgctgccttacctgcaccctgtgttttgatgtgaagtaatctcaacatcagccactttcaagtgttcttaaaatacttcaaagtgttaatacttcttttaagtattcttattctgtgatttttttctttgtgcacgctgtgttttgacgtgaaatcattttgatatcagtgacttttaagtattctttagcttattctgtgatttctttgagcagtgagttatttgaacactgtttatgttcaagatatgtagagtatcaagatacgtagagtattttaagttatcattttattattgatttctaactcagttgtgtagtggtctgtataataccaattatttgaagtttgcggagccttgctttgtgatctagtgtgtgcatggtttccagaactgtccattgtaaatttgacatcctgtcaatagtgggcatgcatgttcactatatccagcttattaaggtccagtgcaaagcttctgtctccttctagatgcatgaaattccaagaaggaggccatagtccctcacctgggggatgggtctgttcatttcttctcgtttggtagcatttatgtgaggcattgttaggtgcatgcacgtggtagaatttttatcttcctgatgagtgaatcttttggagacttctatgtctctagtaatctagtaattctttttttaaattgctcttagtactgccacactgggcttcttttgattagtattttcctgctgtgtctgttttctgcctttaatttatatatatatatatatattttttttttttgagacagagtcttggtctgtcgcccagggtgagtgcagtggtgtgatcacaggtcagtgtaacttttaccttctggcctgagccgtcctctcacctcagcctcctgaatagctggaactgcagacacgcaccgctacacctggctaatttttaaattttttctggagacagggtcttgctgtgttgcccaggctggtctcaaactcttggactcaagggatccatctacctcggcttcccaaagtgctgaattacaggcatgagccaccatgtctggcctaattttcaacacttttatattcttatagtgtgggtatgtcctgttaacagcatgtaggtgaatttccaatccagtctgacagtcgttgtttaactggataacctgatttattttcatttttttgtcactagagacccgcctggtgcactctgattctccacttgcctgttgcatgtcctcgttcccttgtttctcaccacctcttgggttgccatgtgcgtttcctgccgagtgtgtgttgatcctctcgttgcctcctggtcactgggcatttgcttttatttctctttgcttagtgttaccccctgatctttttattgtcgttgtttgcttttgtttattgagacagtctcactctgtcacccaggctggagtgtaatggcacaatctcggctcactgcaacctctgcctcctcggttcaagcagttctcattcctcaacctcatgagtagctgggattacaggcgcccaccaccacgcctggctaatttttgtatttttagtagagataggctttcaccatgttggccaggctggtctcaaactcctgacctcaagtgatctgcccgccttggcctcccacagtgctgggattacaggtgcaagccaccgtgcccggcataccttgatcttttaaaatgaagtctgaaacattgctacccttgtcctgagcaataagacccttagtgtattttagctctggccaccccccagcctgtgtgctgttttccctgctgacttagttctatctcaggcatcttgacacccccacaagctaagcattattaatattgttttccgtgttgagtgtttctgtagctttgcccccgccctgcttttcctcctttgttccccgtctgtcttctgtctcaggcccgccgtctggggtccccttccttgtcctttgcgtggttcttctgtcttgttattgctggtaaaccccagctttacctgtgctggcctccatggcatctagcgacgtccggggacctctgcttatgatgcacagatgaagatgtggagactcacgaggagggcggtcatcttggcccgtgagtgtctggagcaccacgtggccagcgttccttagccagtgagtgacagcaacgtccgctcggcctgggttcagcctggaaaaccccaggcatgtcggggtctggtggctccgcggtgtcgagtttgaaatcgcgcaaacctgcggtgtggcgccagctctgacggtgctgcctggcgggggagtgtctgcttcctcccttctgcttgggaaccaggacaaaggatgaggctccgagccgttgtcgcccaacaggagcatgacgtgagccatgtggataattttaaaatttctaggctgggcgcggtggctcacgcctgtaatcccagcactttgggaggccaaggcgggtggatcacgaggtcaggaggtcgagaccatcctggccaacatgatgaaaccccatctgtactaaaaacacaaaaattagctgggcgtggtggcgggtgcctgtaatcccagctactcgggaggctgaggcaggagaattgcttgaacctgggagttggaagttgcagtgagccgacattgcaccactgcactccagcctggcaacacagcgagactctgtctcaaaaaaaaaaaaaaaaaaaaaaattctagtagccacattaaaaaagtaaaaaagaaaaggtgaaattaatgtaataatagattttactgaagcccagcatgtccacacctcatcattttagggtgttattggtgggagcatcactcacaggacatttgacattttttgagctttgtctgcgggatcccgtgtgtaggtcccgtgcgtggccatctcggcctggacctgctgggcttcccatggccatggctgttgtaccagatggtgcaggtccgggatgaggtcgccaggccctcagtgagctggatgtgcagtgtccggatggtgcacgtctgggatgaggtcgccaggccctgctgtgagctggatgtgtggtgtctggatggtgcaggtcaggggtgaggtctccaggccctcggtgagctggaggtatggagtccggatgatgcaggtccggggtgaggtcgccaggccctgctgtgagctggatgtgtggtgtctggatggtgcaggtcaggggtgaggtctccaggccctcggtgagctggaggtatggagtccggatgatgcaggtccggggtgaggtcgccaggccctgctgtgagctggatgtgtggtgtctggatggtgcaggtctggggtgaggtcaccaggccctgcggtgagctgggtgtgcggtgtctggatggtgcaggtctggagtgaggtcgccagacggtgccagaccgtgcggtgagctggatatgcggtgtccggatggtgcaggtctggggtgaggttgccaggccctgctgtgagttggatgtggggtgtccggatgctgcaggtccggtgtgaggtcaccaggccctgctgtgagctggatgtgtggtgtctggatggtgcaggtctggggtgaggtcgccaggccctgctgtgagctggatgtgtggtgtctggatggtgcaggtctggagtgaggtcgccaggccctcggtgagctggatgtgcagtgtccagatggtgcaggtccggggtgaggtcgccagaccctgcggtgagctggatgtgcggtgtctggatggtgcaggtctggagtgaggtcgccaggccctcggtgagctggatgtatggagtccggatggtgccggtccggggtgaggtcgccagaccctgctgtgagctggatatgcggtgtccggatggtgcaggtcaggggtgaggtctccaggccctcggtgagctggaggtatggagtccggatgatgcaggtccggggtgaggtcgccaggccctgctgtgagctggatgtgcggcgtctggatggtgcaggtctggggtgtggtcgccaggccctcggtgagctggaggtatggagtccggatgatgcaggtccggggtgaggtcgccaggccctgctgtgagctggatgtgcggcgtctggatggtgcaggtctggggtgtggtcgccaggccctcggtgagctggaggtatggagtccggatgatgcaggtccggggtgaggttgccaggccctgctgtgagctggatgtgctgtatccggatggtgcaggtccggggtgaggtcgccaggccctgctgtgagctggatgtgctgtatccggatggtgcaggtctggggtgaggtcaccaggccctgcggtgagctggttgtgcagtgtccggttgctgcaggtccggggtgagttcgccaggccctcggtgagctggatgtgcggtgtccccgtgtccggatggtgcaggtccagggtgaggtcgctaggcccttggtgggctggatgtgccgtgtccggatggtgcaggtctggggtgaggtcgccaggcctttggtgagctggatgtgcggtgtctgcatggtgcatgtctggggtgaggtcgccaggcccttggtgggctggatgtgtggtgtccggatggtgcaggtccggcgtgaggtcgccaggccctgctgtgagctggatgtgcggtgtctggatggtgcaggtccggggtgaggtagccaaggccttcggtgagctggatgtggggtgtccggatggtgcaggtccggggtgaggtcgccaggccctgcggttagctggatatgcggtgtccggatggtgcaggtccggggtgaggtcaccaggccctgcggttagctggatgtgcggtgtctggatggtgcaggtccggggtgaggtcgccaggccctgctgtgagctggatgtgctgtatccagatggtgcaggtccggggtgaggtcgccaggccctgcagtgagctggatgtgctgtatccggatggtgcaggtctggcgtgaggtcgccaggccctgcggttagctggatatgcggtgtcggatggtgcaggtccggggtgaggtcaccaggccctgcggttagctggatgtgtggtgtccggatggtgcaggtctggggtgaggtcgccaggccctgctgtgagctggatgtgctgtatccggatggtgcaggtccggggtgaggtcgccaggccctgcggtgagctggatgtgctgtatccggatggtgcaggtctggcgtgaggtcgccaggccctgcggtgagctggatgtgcagtgtacggatggtgcaggtccggagtgaggtcgccaggccctgcggtgggctgtatgtgtgttgtctggatggtgcaggtccggggtgagttcgccaggccctgcggtgagctggatgtgtggtgtctggatgctgcaggtccggggtgagttcgccaggccctcggtgagctggatatgcggtgtccccgtgtccgaatggtgcaggtccagggtgaggtcgccaggcccttggtgggctggatgtgccgtgtccggatggtgcaggtctggggtgaggtcgccaggcccttggtgagctggatgtgcggtgtccggatggtgcaggtccggggtgaggtcaccaggccctcggtgatctggatgtggcatgtccttctcgtttaaggggttggctgtgttccggccgcagagcaccgtctgcgtgaggagatcctggccaagttcctgcactggctgatgagtgtgtacgtcgtcgagctgctcaggtctttcttttatgtcacggagaccacgtttcaaaagaacaggctctttttctaccggaagagtgtctggagcaagttgcaaagcattggaatcaggtactgtatccccacgccaggcctctgcttctcgaagtcctggaacaccagcccggcctcagcatgcgcctgtctccacttgcctgtgcttccctggctgtgcagctctgggctgggagccaggggccccgtcacaggcctggtccaagtggattctgtgcaaggctctgactgcctggagctcacgttctcttacttgtaaaatcaggagtttgtgccaagtggtctctagggtttgtaaagcagaagggatttaaattagatggaaacactaccactagcctccttgcctttccctgggatgtgggtctgattctctctctcttttttttttcttttttgagatggagtctcactctgttgcccaggctggagtgcagtggcataatcttggctcactgcaacctccacctcctgggtttaagcgattcaccagcctcagcctcctaagtagctgggattacaggcacctgccaccacgcctggctaatttttgtacttttaggagagacggggtttcaccatgttggccaggctggtctcgaactcatgacctcaggtgatccacccaccttggcctcccaaagtgctgggtttacaggctaagccaccgtgcccagcccccgattctcttttaattcatgctgttctgtatgaatcttcaatctattggatttaggtcatgagaggataaaatcccacccacttggcgactcactgcagggagcacctgtgcagggagcacctggggataggagagttccaccatgagctaacttctaggtggctgcatttgaatggctgtgagattttgtctgcaatgttcggctgatgagagtgtgagattgtgacagattcaagctggatttgcatcagtgagggacgggagcgctggtctgggagatgccagcctggctgagcccaggccatggtattagcttctccgtgtcccgcccaggctgactgtggagggctttagtcagaagatcagggcttccccagctcccctgcacactcgagtccctggggggccttgtgacaccccatgccccaaatcaggatgtctgcagagggagctggcagcagacctcgtcagaggtaacacagcctctgggctggggaccccgacgtggtgctggggccatttccttgcatctgggggagggtcagggctttccctgtgggaacaagttaatacacaatgcaccttacttagactttacacgtatttaatggtgtgcgacccaacatggtcatttgaccagtattttggaaagaatttaattggggtgaccggaaggagcagacagacgtggtggtccccaagatgctccttgtcactactgggactgttgttctgcctggggggccttggaggcccctcctccctggacagggtaccgtgccttttctactctgctgggcctgcggcctgcggtcagggcaccagctccggagcacccgcggccccagtgtccacggagtgccaggctgtcagccacagatgcccaggtccaggtgtggccgctccagcccccgtgcccccatgggtggttttgggggaaaaggccaaagggcagaggtgtcaggagactggtgggctcatgagagctgattctgctccttggctgagctgccctgagcagcctctcccgccctctccatctgaagggatgtggctctttctacctgggggtcctgcctggggccagccttgggctaccccagtggctgtaccagagggacaggcatcctgtgtggaggggcatgggttcacgtggccccagatgcagcctgggaccaggctccctggtgctgatggtgggacagtcaccctgggggttgaccgccggactgggcgtccccagggttgactataggaccaggtgtccaggtgccctgcaagtagaggggctctcagaggcgtctggctggcatgggtggacgtggccccgggcatggccttcagcgtgtgctgccgtgggtgccctgagccctcactgagtcggtgggggcttgtggcttcccgtgagcttccccctagtctgttgtctggctgagcaagcctcctgaggggctctctattgcagacagcacttgaagagggtgcagctgcgggagctgtcggaagcagaggtcaggcagcatcgggaagccaggcccgccctgctgacgtccagactccgcttcatccccaagcctgacgggctgcggccgattgtgaacatggactacgtcgtgggagccagaacgttccgcagagaaaagagggtggctgtgctttggtttaacttcctttttaaacagaagtgcgtttgagccccacatttggtatcagcttagatgaagggcccggaggaggggccacgggacacagccagggccatggcacggcgccaacccatttgtgcgcacagtgaggtggccgaggtgccggtgcctccagaaaagcagcgtgggggtgtagggggagctcctggggcagggacaggctctgaggaccacaagaagcagccgggccagggcctggatgcagcacggcccgaggtcctggatccgtgtcctgctgtggtgcgcagcctccgtgcgcttccgcttacggggcccggggaccaggccacgactgccaggagcccaccgggctctgaggatcctggaccttgccccacggctcctgcaccccacccctgtggctgcggtggctgcggtgaccccgtcatctgaggagagtgtggggtgaggtggacagaggtgtggcatgaggatcccgtgtgcaacacacatgcggccaggaacccgtttcaaacagggtctgaggaagctgggaggggttctaggtcccgggtctgggtggctggggacactggggaggggctgcttctcccctgggtccctatggtggggtgggcacttggccggatccactttcctgactgtctcccatgctgtccccgccaggccgagcgtctcacctcgagggtgaaggcactgttcagcgtgctcaactacgagcgggcgcggcgccccggcctcctgggcgcctctgtgctgggcctggacgatatccacagggcctggcgcaccttcgtgctgcgtgtgcgggcccaggacccgccgcctgagctgtactttgtcaaggtgggtgccggggacccccgtgagcagccctgctggaccttgggagtggctgcctgattggcacctcatgttgggtggaggaggtactcctgggtgggccgcagggagtgcaggtgaccctgtcactgttgaggacacacctggcacctagggtggaggccttcagcctttcctgcagcacatggggccgactgtgcaccctgactgcccgggctcctattcccaaggagggtcccactggattccagtttccgtcagagaaggaaccgcaacggctcagccaccaggccccggtgccttgcaccccagtcctgagccaggggtctcctgtcctgaggctcagagaggggacacagcccgccctgcccttggggtctggagtggtgggggtcagagagagagtgggggacaccgccaggccaggccctgagggcagaggtgatgtctgagtttctgcgtggccactgtcagtctcctcgcctccactcacacaggtggatgtgacgggcgcgtacgacaccatcccccaggacaggctcacggaggtcatcgccagcatcatcaaaccccagaacacgtactgcgtgcgtcggtatgccgtggtccagaaggccgcccatgggcacgtccgcaaggccttcaagagccacgtaaggttcacgtgtgatagtcgtgtccaggatgtgtgtctctgggatatgaatgtgtctagaatgcagtcgtgtctgtgatgcgtttctgtggtggaggtacttccatgatttacacatctgtgatatgcgtgtgtggcacgtgtgtgtcgtggtgcatgtatctgtggcgtgcatatttgtggtgtgtgtgtgtgtggcacgtgtgtgtccatggtgtgtgtgcctgtggtgtgcatgtgtgtgtgtctgtgacacgtgcatgttcatgctgtgtgctgcatgtctgtgatgtgcctatttgtggtgtgtgtgtgtgcatgtgtccgtgacatatgcgtgtctatggcatgggtgtgtgtggccccttggccttactccttcctcctccaggcatggtccgcaccattgtcctcacgctctcgggtgctggtttggggagctccacattcagggtcctcacttctagcatgggtgcccctgtcctgtcacagggctgggccttggagactgtaagccaggtttgagaggagagtagggatgctggtggtaccttcctggacccctggcacccccaggaccccagtctggcctatgccggctccatgagatataggaaggctgattcaggcctcgctccccgggacacactcctcccagagcggccgggggccttggggctcggcaggggtgaaaggggccctgggcttgggttcccacccagtggtcatgagcacgctggaggggtaagccctcaaagtcgtgccaggccggggtgcagaggtgaagaagtatccctggagcttcggtctggggagaggcacatgtggaaacccacaaggacctctttctctgacttcttgagctatcgcagctacttgcaaatctcagcagaattttaccctcatggaaagctgatggccaccacctgtgcccaggtgtagaaagtcactagagcccaggagacccccccgtccccaccgcccacctgtgcccgggtgtagaaagtcaccagagcccaggagacccccccccacatccccgctgcagcggaagctaccgcccccaccgtgaccctcgttccgacctcgacctccatggtctccctctcccggtctccatgtgagtgagatcaggcaggaggtgggttttggggttcgagcctcagctatgctgaacgcctcagctgtgcgttctgtccatgaatggcatccgttgtggaactcgccacgtggatgtatccttcccgctgctgtggaggctcctgggtgcggactgcacggctgcgcctgctgtgagcctgctgggttctgggagtgtcccaggggtcagaggccctcgcctctgggtgaattccaggggtggggttgctgggtcacagacgtgctttagggatgttgtgggagtttccaggggggtgacagtgacaccccggctggctgcttgtgagggcgatgcctccggtggccgctggcctgaaggtgtcgaagccgcctcgtgggattgtgcagggggctctcttgatgcccacgtgcccacagtgttcccgtgctggccgccccggggctcttgcgggaggtggctgtccgtcctgccccgctctgtatggggcgcctgcctgccatcatcggcttgcaggagctctttgtgtgttctgctgcaagtgtggcatcacatgcgtgcactgtgaatctctgtgccgtgtgtgtcttgcttttgtgctctcaagtcaagccttttgaggactggaagattgtacttctatcgcagtccactaacacagtccagtaacgcagtccactttatcagctgtttgtggctaaagctgtttgtgttctctgcaggaactcaaggtattgaggttgtggtgacgttgcttctgcgcctccttttagaacagtctttactgtgtcagcttgcaaacctgggtggggattggtggaattggttttcatgtgtggggtaggtggggatctgtgggattggttttcatgtgtggtgtaggtggagatctgtgaattggttttcatccgtggggtaggtggggatctgtgggattggtttttatgtgtggggtaggtgggattggttttcatgtgcggggtaggtggggatctgtgggattggttttcttgtgtggggtaggtggggatctgtgggattggtttttatgtgtggggtaggtggggatatgtgaattggttttcatccgtggggtaggtggggatctgtgggattggtttttatgcgtggggtaggtggggatctgtgaattggttttcatccgtggggtaggtggggatctgtgggattggttttcatgtgtgggataggtggggatctgtggagttggttttcatgtgtggtgtaggtggagatctgtgaattggttttcatccgtggggtaggtggggatctgtgggattggtttctatgtgtggggtaggtggggacctgtgggattggtttttatgtgtggggtaggtggggatctgtgaattggttttcatccgtggggtaggtggggatctgtgggattggttttcatgtgtggggtaggtggggatctgtgggattggttttcatgtgtggggtaggtggggatctgtgggattggttttcatgtgtggggtaggtggggatctgtggagttggtttttatgtgtgggggtaggtggggatctgtggagttggttttcatgtgtagggtaggtggagatctgtgggattggttttcatgtgtggggtaggtggggatctgtgggattggttttcatgtgtggggtaggtggggatctgtgggattggttttcatgtgtggggtaggtggggatctgtggagttggttttcatgtgtggggtaggtggagatctgtgggattggttttcatgtgtgggataggtggggatctgtgggattggtttttatgagtggggtaacacagagttcaaggcgagctttcttcctgtagtgggtctgcaggtgctccaacagctttattgaggagaccatatcttcctttgaactatggtcgggtttatagtaagtcaggggtgtggaggcctcccctgggctccctgttctgtttcttccactctggggtcgtgtggtgcctgctgtggtgtgtggccggtgggcagggcttccaggcctccttgtgttcattggcctggatgtggccctggctacgctccgtccttggaattcccctgcgagttggaggcttgctttctttctttttttctttctttttttttttttttgataacagagtctcgctcttttttgcccaggctggagtggtttggcgtgatcttggctcactgcaacctgtgcttcctgagttcaagcaattctcttgcctcagcctcccaagtagctggaattataggcgcccaccaccatgctgactaatttttgtaattttagtagagacgaggtttctccatgttggccaggctggtctcgaactcctgacctcaggtgatcctcccacctcggcctcccaaagtgctgggatgacaggtgtgaaccgccgcgcccggccgagactcgcttcctgcagcttccgtgagatctgcagcgatagctgcctgcagccttggtgctgacaacctccgttttccttctccaggtctcgctaggggtctttccatttcatgactctcttcacagaagagtttcacgtgtgctgatttcccggctgtttcctgcgtaattggtgtctgctgtttatcgatggcctccttccatttcctttaggctttgtttattgttgtttttccggctccttgaaggaaaagtttcgattatggatgtttgaactttcttttctaaacaagcatctgaagttgccgttttccctctaaagcagggatcccgaggcccctggctgtggagtggcaccggtctggggcctgttaggaacccggcgcacagcgggaggctaggtggggtgtggggagccagcgttcccgcctgagccccgcccctctcagatcagcagtggcatgcggtgctcagaggcgcacacaccctactgagaactgtgcgtgagaggggtctagattctgtgctccttatgggaatctaatgcctgatgatctgaggtggaaccgtttgctcccaaaaccatccccttccccactgctgtcctgtggaaaaatcgtcttccacgaaaccagtccctggtaccacaatggttggggaccctgtgctaaagacctgcttcagcagcctctcgtcagtgttgatatattggcttttctgtgttgagtccagaataattacggatttctgtgatgctttccgccgacctcagacccatgggctatttgtgggcgtgttgcctgctcctgggttgggaagggtgcaggccccatgtaccttcctgttactgccttccaggttggttctcagggttgaatcgtactcgatgtggttttagcccacggccctgccgccagctcctgggggctggggaacatgctgaagcacagagtcaccgtgcgcgtcttttgatgcctcacaagctcgaggcctcctgtgtccgtgttagtgtgtgtcacgtgcctgctcacatcctgtcttggggacgcaggggcttagcaggtcccgtagtaaatgacaagcgtcctgggggagtctgcagaataggaggtgggggtgccggtctctctcccgcgtcttcagactcttctcctgcctgtgctgtggctgcacctgcatccctgcaatccctccagcactgggctggagaggcccgggagctcgagtgccacttgtgccacgtgactgtggatggcagtcggtcacgggggtctgatgtgtggtgactgtggatggcggttggtcacaggggtctgatgtgtggtgactgtggatggcggtcgtggggtctgatgtggtgactgtggatggcggtcgtggggtctgatgtggtgactgtggatggcggtcgtggggtctgatgtgtggtgactgtggatggcggttggtcccgggggtctgatgtgtggtgactgtggatggcggtcgtggggtctgatgtggtgactgtggatggcggtcgtggggtctgatgtggtgactgtggatggcggtcgtggggtctgatgtgtggtgactgtggatggcggttggtcccgggggtctgatgtgtggtgactgtggatggcgatcggtcacaggggtctgatgtgtggtgactgtggatggcggtcgtggggtctgatgtgtggtgactgtggatggcggtcgtggggtctgatgtggtgactgtggatggcggtcgtggggtctgatgtgtggtgactgtggatggcggtcgtggggtctgatgtggtgactgtggatggcggtcgtggggtctgatgtgtggtgactgtggatggcggtcgtggggtctgatgtgtggtgactgtggatggcggtcgtggggtctgatgtgtggtgactgtggatggcggtcgtggggtctgatgtggtgactgtggatggcggtcgtggggtctgatgtgtggtgactgtggatggcggttgtggggtctgatgtgtggtgactgtggatggtgatcggtcacaggggtctgatgtgtggtgactgtggatggcggtcgtggggtctgatgtgtggtgactgtggatggcggttggtcccgggggtctgatgtgtggtgactggatggcgatcggtcacaggggtctgatgtgtggtgactgtggatggcggtcgtggggtctgatgtgtggtgactgtggatggcggtcgtggggtctgatgtggtgactgtggatggcggtcgtggggtctgatgtgtggtgactgtggatggcggtcgtggggtctgatgtggtgactgtggatggcggtcgtggggtctgatgtgtggtgactgtggatggcggtcgtggggtctgatgtgtggtgactgtggatggcggtcgtggggtctgatgtgtggtgactgtggatggcggtcgtggggtctgatgtgtggtgactgtggatggcggtcgtggggtctgatgtggtgactgtggatggcggtcgtagggtctgatgtggtgactgtggatggcggtcgtggggtctgatgtggtgactgtggatggtgatcggtcacaggggtctgatgtgtggtagctgcaggtggagtcccaggtgtgtctgtagctactttgcgtcctcggccccccggcccccgtttcccaaacagaagcttcccaggcgctctctgggcttcatcccgccatcgggcttggccgcaggtccacacgtcctgatcggaagaaacaagtgcccagctctggccggggcaggccacatttgtggctcatgccctctcctctgccggcaggtctctaccttgacagacctccagccgtacatgcgacagttcgtggctcacctgcaggagaccagcccgctgagggatgccgtcgtcatcgagcaggtctgggcactgccctgcagggttgggcacggactcccagcagtgggtcctcccctgggcaatcactgggctcatgaccggacagactgttggccctggggggcagtggggggaatgagctgtgatgggggcatgatgagctgtgtgccttggcgaaatctgagctgggccatgccaggctgcgacagctgctgcattcaggcacctgctcacgtttgactgcgcggcctctctccagttccgcagtgcctttgttcatgatttgctaaatgtcttctctgccagttttgatcttgaggccaaaggaaaggtgtccccctcctttaggagggcaggccatgtttgagccgtgtcctgcccagctggcccctcagtgctgggtctgaggccaaaggaaacgtgtcccccttcttaggaggacgggccgtgtttgagccacgccccgctgagcgggcctctcagtgctgggtctgtccacgtggccctgtggccctttgcagatgtggtctgtccacgtggccctgtggctctttgcagatgcctgttagcacttgctcggctctaggggacagtcgtgtccaccgcatgaggctcagagacctctgggcgaatttccttggctcccagggtgggggtggaggtggcctgggctgctgggacccagaccctgtgcccggcagctgggcagcaactcctggatcacatatgccatccgggccacggtgggctgtgtgggtgtgagcccagctggacccacaggtggcccagaggagacattctgtgtcacacactctgcctaagcccatgtgtgtctgcagagactcggcccggccagcccacgatggccctgcattccagcccagccccgcacttcatcacaaacactgaccccaaaagggacggagggtcttggccacgtggtcctgcctgtctcagcacccaccggctcactcccatgtgtctcccgtctgctttcgcagagctcctccctgaatgaggccagcagtggcctcttcgacgtcttcctacgcttcatgtgccaccacgccgtgcgcatcaggggcaagtgagtcaggtggccaggtgccattgccctgcgggtggctgggcgggctggcagggcttctgctcacctctctcctgccccttccccactgcccttctgcccggggccaccagagtctccttttctggcccccgccccctccggctcctgggctgcaggctcccgaggccccggaaacatggctcggcttgcggcagccggagcggagcaggtgccacacgaggcctggaaatggcaagcggggtgtggagttgctcctgcgtggaggacgaggggcggggggtgtgtctgggtcaggtgtgcgccgagcgtttgagcctgcagcttgtcagctccaagttactactgacgctggacacccggctctcacacgcttgtatctctctctcccgatacaaaaggattttatccgattctcattcctgtccctgtcgtgtgacccccgcgagggcgcgggctcttctctctgtgactagatttcccatctggaaagtgcggggttgaccgtgtagtttgctcctctcggggggcctgtggtggccatggggcaggcggcctgggagagctgccgtcacacagccactgggtgagccacactcacggtggtagagccacagtgcctggtgccacatcacgtcctctggattttaagtaaaaccacacacctcccggcaggcatctgcctgcgaccctgtgtgtgcctggggagagtggtagcacggaggaaattcgtgcacactcaaggtcatcagcaaggtcatccgcagtcaggtggaacgtggaggcctctctctgggatcgtctccagcggataaaggactgtgcacagcttcggaagcttttatttaaaaatataactattaattattgcattataagtaatcactaatggtatcagcaattataatatttattaaagtataattagaaatattaagtagtacacacgttctggaaaaacacaaattgcacatggcagcagagtgaattttggccgagggacacgtgtgcacatgtgtgtaagcggcccccaggcccacagaattcgctgacaaagtcacctccccagagaagccaccacgggcctccttcgtggtcgtgaattttattaagatggatcaagtcacgtaccgtccacgtgtggcagggctttggggaatgtgaggtgatgactgcgtcctcatgccctgacagacaggaggtgactgtgtctgtcctgtccctaggacacggacaggcccgaagctctagtccccatcgtggtccagtttggcctctgaataaaaacgtcttcaaaacctgttgccccaaaaactaagaacagagagagtttcccatcccatgtgctcacaggggcgtatctgcttgcgttgactcgctgggctggccggactcctagagttggtgcgtgtgcttctgtgcaaaaagtgcagtcctcttgcccatcactgtgatatctgcaccagcaaggaaagcctcttttcttttctttctttttttttttttgagacggaacgtcactgttgtctgcctgggcttgagtgcagtggcgcgatctcaactcactgcaacctccgcctcccgggttccagcatttctcctgcctcagcctcccgagcagctgagattacaggcacccaccccctgcgcctggctaatttttgtatttttagtagagaggggtttttgccatgttggccaggctggtctcgaactcctgacctcaggtgatccacccacctcggcctcccaaagtgctgggattacaggtgtgagccatcacgcccagccggaaagcctctttttaaggtgaccacctatagcgcttcccgaaaataacaggtcttgtttttgcagtaggctgcaagcgtctcttagcaacaggagtggcgtcctgtgggctctggggatggctgagggtcgcgtggcagccatgccttctgtgtgcacctttaggttccacggggctattctgctctcactgtttgtctgaaaacgcacccttggcatccttgtttggagagtttctgcttctcgttggtcatgctgaaactaggggcaaggttgtatccgttggcgcgcagcggctacatgtagggtcatgagtctttcaccgtggacaaattccttgaaaaaaaaaaaaggagtccggttaagcattcattccgggtcaagtgtctggttctgtgaataaactctaagatttaagaaaccttaatgaaagaaaaccttgatgattcagagcaaggatgtggtcacacctgtggctggatctgtttcagccgccccagtgcatggtgagagtggggagcagggattgtttgttcagaggtctcatctggtatgtttctgaggtgtttgccggctgaatggtagacgtgtcgtttgtgtgtatgaggttctgtgtctgtgtgtggctcggtttgagtgtacgcatgtccagcacatgccctgcccgtctctcacctgtgtcttcccgccccaggtcctacgtccagtgccaggggatcccgcagggctccatcctctccacgctgctctgcagcctgtgctacggcgacatggagaacaagctgtttgcggggattcggcgggacgggtgaggcctcctcttccccaggggggcttgggtgggggttgatttgcttttgatgcattcagtgttaatattcctggtgctctggagaccatgactgctctgtcttgaggaaccagacaaggttgcagccccttcttggtatgaagccgcacgggaggggttgcacagcctgaggactgcgggctccacgcaggctctgtccagcggccatgtccagaggcctcagggctcagcaggcgggagggccgctgccctgcatgatgagcatgtgaattcaacaccgaggaagcacaccagcttctgtcacgtcacccaggttccgttagggtccttggggagatggggctggtgcagcctgaggccccacatctcccagcaggccctcgacaggtggcctggactgggcgcctcttcagcccattgcccatcccacttgcatggggtctacacccaaggacgcacacacctaaatatcgtgccaacctaatgtggttcaactcagctggcttttattgacagcagttacttttttttttttaatactttaagttctagggtacatgtgcacgacgtgcaggttagttacatatgtatacatgtgccatgttggtgtgctgcacccattaactcatcatttacattaggtatatctcctaatgctatccctccccactccccccatcccatgacaggccctggtgtgtgatgttccccaccctgtgtccaagtgttctcattgttcagttcccacctgtgagtgagaacatgtggtgtttggttttctttccttgcaatagtttgctcagagtgatggtttccagcttcgtccatgtccctacaaaggacatgaactcatccttttttatgactgcatagtattccgtggtgtatatgtgccacattttcttaatccagtctatcatcgatggacatttgggttggttgcaagtctttgctactgtgaatagtgccgcaataaacatacgtgtgcatgtgtctttatagcagcatgatttataatcctttgggtatatacccagtaatgggatggctgggtcaaatggtatttctagttctagatccttgaggaatcaccacactgtcttccacaatggttgaactagtttacactcccaccaacagtgtaaaagtgttctggtgctggagaggatgtggacagcagttatttttttatgaaaatagtatcactgaacaagcagacagttagtgaaggatgcgtcaggaagcctgcaggccacacagccatttctctcgaagactccgggtttttcctgtgcatcttttgaaactctagctccaattatagcatgtacagtggatcaaggttcttcttcattaaggttcaagttctagattgaaataagtttatgtaacagaaacaaaaatttcttgtacacacaacttgctctgggatttggaggaaagtgtcctcgagctggcggcacactggtcagccctctgggacaggatacctctggcccatggtcatggggcgctgggcttgggcctgagggtcacacagtgcaccatgcccagcttcctgtggataggatctgggtctcggatcatgctgaggaccacagctgccatgctggtaaagggcaccacgtggctcagagggggcgaggttcccagccccagctttcttaccgtcttcagttatttttccctaagagtctgagaagtggggccgcgcctgatggccttcgttcgtcttcagctggcacagaattgcacaagctgatggtaaacactgagtacttataatgaatgaggaattgctgtagcagttaactgtagagagctcgtctgttggaaagaaatttaagtttttcatttaaccgctttggagaatgttactttatttatggctgtgtaaattgtttgacattcagtccctcgtagacagatactacgtaaaaagtgtaaagttaaccttgctgtgtattttcccttattttaggctgctcctgcgtttggtggatgatttcttgttggtgacacctcacctcacccacgcgaaaaccttcctcaggtgaggcccgtgccgtgtgtctgtggggacctccacagcctgtgggctttgcagttgagccccccgtgtcctgcccctggcaccgcagcgttgtctctgccaagtcctctctctctgccggtgctggatccgcaagagcagaggcgcttggccgtgcacccaggcctgggggcgcaggggcaccttcgggagggagtgggtaccgtgcaggccctggtcctgcagagacgcacccaggttacacacgtggtgagtgcaggcggtgacctggctcctgctgctctttggaaagtcaagagtggcggctcctggggccccagtgagacccccaggagctgtgcacagggcctgcagggccgaggcggcagcctcctccccagggtgcacctgagcctgcggagagcaggagctgctgagtgagctggcccacagcgttcgctgcggtcacgttcctgcgtggggttgtttgggatcggtgggagaatttggatttgctgagtgctgctgtcttgaaccacggagatggctaggagtgggtttcagagttgatttttgtgaatcaaactaaaatcaggcacaggggacctggcctcagcacaggggattgtccaatgtggtccccctcaagggcgccccacagagccggtgggcttgttttaaagtgcgatttgacgagggacgagaaaccttgaaagctgtaaagggaaccctcagaaaatgtggccgccaggggtggtttcaggtgctttgctgggctgtgtttgtgaaaacccatttggacccgccctccaagtccaccctccaggtccaccctccagggccgccctgggctgggggtatgcctggcgttccttgtgccgcagcccggagcacagcaggctgtgcacatttaaatccactaagattcactcggggggagcccaggtcccaagcaactgagggctcaggagtcctgaggctgctgaggggacagagcagacggggaacgctgcttctgtgtggcaagttcctgagggtgctggccagggaggtggctcagagtgtatgttggggtcccaccgggggcagaactctgtctctgatgagtcggcagccatgtaacaggaaggggtggccacagggagctgggaatgcaccaggggagctgcgcagctggccgaggtcccagggccaggccacaggaagggcagggggacgcccggggccacagcagaggccgcaggaagggaaggggatgcccaggccagagcagaggctaccgggcacaggggggctccctgagctgggtgagcgaggctcatgactcggcgagggaacctccttgacgtgaagctgacgactggtgttgcccagctcacagcccagccaggtcccgcgcctgagcaggaactcagaaccctcccctttgtctaaagcacagcagatgccttcagggcatctaggagaaaacaggcaaagtcgttgagaaacgtcttaaaagaaggtgggatggtggcaatttcttgtccagattttagtctgccccggaccacagatgagtctataacgggattgtggtgttgccatggggacacatgagatggaccatcacagaggccactggggctgcacctcccatctgagtcctggctgtcccgggtccaggccaggttcttgcatgctcacctacctgtcctgcccgggagacagggaaagcaccccgaagtctggagcagggctgggtccaggctcctcagagctcctgccaggcccagcaccctgctccaaatcaccacttctctggggttttccaaagcatttaacaagggtgtcaggttacctcctgggtgacggccccgcatcctggggctgacattgcccctctgccttaggaccctggtccgaggtgtccctgagtatggctgcgtggtgaacttgcggaagacagtggtgaacttccctgtagaagacgaggccctgggtggcacggcttttgttcagatgccggcccacggcctattcccctggtgcggcctgctgctggatacccggaccctggaggtgcagagcgactactccaggtgagcgcacctggccggaagtggagcctgtgcccggctggggcaggtgctgctgcagggccgttgcgtccacctctgcttccgtgtggggcaggcgactgccaatcccaaagggtcagaggccacagggtgcccctcgtcccatctggggctgagcagaaatgcatctttctgtgggagtgagggtgctcacaacgggagcagttttctgtgctattttggtaaaaggaaatggtgcaccagacctgggtgcactgaggtgtcttcagaaagcagtctggatccgaacccaagacgcccgggccctgctgggcgtgagtctctccaaacccgaaacacaggggccctgctgggcatgagtccctctgaacccgagaccctggggccctgctgggcgtgagtctctccgaacccagagacttcagggcccttttgggcgtgagtctctccgctgtgagccccacactccaaggctcatccacagtctacaggatgccatgagttcatgatcacgtgtgacccatcaggggacagggccatggtgtggggggggtctctacaaaattctggggtcttgtttccccagagcccgagagctcaaggccccgtctcaggctcagacacaaatgaattgaagatggacacagatgcagaaatctgtgctgtttcttttatgaataaaaagtatcaacattccaggcagggcaaggtggctcacacctataatcccagcactttgggaggccgaggtgggtggatcacttgaggccaggagtttgaggccaacctaaccaacatagtgaaattccatttctacttaaaaaatacaaaaattagcctggcctggtggcacacgcctgtagtccccgctatgcgggaggctgaggcaggagaatcatttgaacccaggaggcagaggttgcagtgagccgagatcacaccactgcactccagcctgggcaacagagtgagacttcatcttaaaaaaaaaaaaaaaagtatcagcattccaaaaccatagtggacaggtgtttttttattctgtccttcgataatatttactggtgctgtgctagaggccggaactgggggtgccttcctctgaaaggcacaccttcatgggaagagaaataagtggtggatggttgttaaaccagaggtttaaactggggtcctgtcgttctgagttaacagtccagatctggactttgcctctttccagaatgctccctggggtttgcttcatgggggagcagcaggtgtggacaccctcgtgatgggggagcagcaggtgcagacgccctcatgatgggggagtggcaggtgcagacacccttgtgcatggtgcccagcatgtccctgttgcagctccctccccacaaggatgccggtctcctgtgctccccacagtccctgcttccctctcacagccttacctggtcctggcctccactggctttgtctgcatgatttccacatttcctgggctcccagcacctcttcgcctctcccaggcacctctgcagtgctggccataccagtcagctgtgaactgtccactgcttattttgctccccatgaaatgtattttttaggacaggcacccctggttccagcctctggcacagcatcagtgaatgttattgaaggacaaaggacagacaaacaaatcaggaaaatgggttctctctaaacacattgcaaagccacagaggctagtgcaggatgggtgggcatcaggtcatcagatgtgggtccaatgccagaatattctgtgctcccaaaggccacttggtcagagtgtgtgcttgcagaggtggctctaaaagctcagcagtggaggcagtggttcgccatactcagggtgaactcacatcctctgtgtctgaagtatacagcagaggcttgaagggcatctgggagaagaaaacaggcaaaatgattaagaaaagtgaaaaaggaaaagtggtaagatgggaattttcttgtccagattttagtctcccaaaccacagctcagatggtagaatgtggtcagaactgatggacagaacaatagaacaaaacggaagccctatctctcagaaacgtgtgttaatgtggtatgtggcacagctgatggaaaagagagtgtgtgtgtaatttttttttctgagaaaactgactggaagcaaataagttgtgtctttacagcatataccagagcagattctaggtagaagaggagacacatgcaaacaacaccagcaacagaaataaaacaaaagactcaaagggaagggaggtgaacgttccctggtttggtgttggggaaggacacacagggaggcggatgaaaccagtgaggcaacgggcattgctttcactgcagagaaactcagcttgcctgagccacagtgaaaatggccattccctggagcgtttgtgcacgtgatttatttaaggcgccctgtgaggtcctgcacattcatcctctcactttgttctcctaaccacctgagaggtagaggaggaaaggctccaggggagcagccgcccttggtcacccagctggcaaagggcatgcatgattgcagcctggcctcctgctccggggcccttgctctgcccgaggaccccacacaagtcagacccataggctcagggtgagccggagcccaaggtcgtgttggggatggctgtgaaagaagaaatggacgtctgatgcacacttgggaaggtcctaccagcagcgtcaaagaaatgcatgtgaaactgacagcgagacccatccctcaaagaaacgcacgtgaaactgatggcgagacctgtccccatccctcatgctggctccttttctgggcttgccaagagccagcatcaggttgaggcaagctggaaagacttttctggaaagcagcttgtttgcatggaagtcctcacaatgtcctgtgtcttcccagtaattccacttctgaagtgaccagacattatcacgggtcttatttaccatttccagtgttccaggcagggggacttgccacagcaagtcacgaacctgcccaaatacagggctaaggagatattatgcatcacaaaacttgctctgccattaaacatttttcaaagaatttttgaagaatgtttaatggcacaaaacgtttatttcaatgtagcagtgttcaaagctggatgtaaaagaacacaccccaggagcctgccgtggatgtcatgtgtgttcatctttggacatggacatacatgggcagtgagtggtggtgaggccctggaggacatcggtgggatgcctccatcctgcccctctggagacaccatgtgtgccacgtgcactcactggagccctgtttagctggtgccacctggctcttccatccctgagattcaaacacagtgagattccccacgcccaactcagtgttctcccacaaaaaacctgagtcacacctgtgttcactcgagggacgcccgggagccagggctccacagtttattatgtgtttttggctgagttatgtgcagatctcatcagggcagatgatgagtgcacaaacacggccgtgcgaggtttggatacactcaacatcactagccaggtcctggtggagtttggtcatgcagagtctggatggcatgtagcatttggagtccatggagtgagcacccagccccctcgggctgcagcgcatgccccaggcaggacaaggaagcgggaggaaggcaggaggctctttggagcaagctttgcaggagggggctgggtgtggggcaggcacctgtgtctgacattcccccctgtgtctcagctatgcccggacctccatcagagccagtctcaccttcaaccgcggcttcaaggctgggaggaacatgcgtcgcaaactctttggggtcttgcggctgaagtgtcacagcctgtttctggatttgcaggtgagcaggctgatggtcagcacagagttcagagttcaggaggtgtgtgcgcaagtatgtgtgtgtgtgtgtgcgcgcgtgcctgcaaggctgatggtgactggctgcacgtaagagtgcacatgtacgcatatacacgtgagcacatacatgtgtgcatgtgtgtacatgaaggcatggcagtgtgtgcacaggtgtgcaagggcacaagtgtgtgcacatgcgaatgcacacctgacatgcatgtgtgttcgtgcacagtcgtgtgggcattcacgtgaggtgcatgcgtgtgggtgtgcagtgtgagtagcatgtgtgcacataacatgtattgaggggtcctcgtgttcaccccgctaggtcctcagcaccagtgccactccttacaggatgagacggggtcccaggccttggtgggctgaggctctgaagctgcagccctgagggcattgtcccatctgggcatccgcgtccactccctctcctgtgggcttctgtgtccactccccctctcctgtgggcgtccacaccccctctcctgtgggcatccgcatccactccctctcctgtgggcatctgtgtccactccctctctcctgtggttgtccgtgtccactcccccctcctgtgggcgtccgcgtccactccctctcctgtgggcatctgcatccactccctctctcctgtgggcgtctgtgtccactccccctctcctgtgggcatccgcgtccactccctcttctgtgggcatctgcatccactcgtctctcctgtgggcgtccgtgtccactccccctctcctgtgggcatctgcgtccactccctctctcctgtgggcatccgtgtccactccccctctcctgtgggcatccatgtccactccctctcctgtgggcatctgcgtccactccctctcctgtgggcatctgcgtctgctccctctctcctgtgggcgtccgtgtccactcccccctcctgtgggcgtccgcgtccactccctctcctgtgggcatctgcgtccgctccctctctcctgtgggcgtccgtgtccactcccccctcctgtgggcgtccgcatccactccctctcctgtgggcatctgcgtctgctccctctctcctgtgggcgtccatgtccactccctctcctgtgggcatctgcgtccactccctctcctgtgggcatccgcgtccactccctctcctgtgggcatctgcatccactccatctcctgtgggcatccacgtccactccctctcctgtgtgcatctgcatccactccactccccctctcctgtgggcatccgcgtccactccctctcctgtgggcatctgcatccactccctctctcctgtgggcgtccgtgtccactccccctctcctgtgggcatttacatccactccattccctctctcctgtgggcgtctgtgtccactccccctctcctgtgggcatttacatccactccactccctctctcctgtgggcatccgcgtccactccccctctctgtgggcatctgcgtccacctcccctctctgtgggcatttgcgtccactccctctcctggttccttcctgtcttggccgagcctcgggggcaggcagatgacacagagtcttgactcgcccagggtggttcgcagctgccgggtgagggccaggccggatttcactgggaagagggatagtttcttgtcaaaatgttcctctttcttgttccatctgaatggatgataaagcaaaaagtaaaaacttaaaatcccagagaggtttctaccgtttctcactctttcttggcgactctaggtgaacagcctccagacggtgtgcaccaacatctacaagatcctcctgctgcaggcgtacaggtgagccgccaccaaggggtgcaggcccagcctccagggaccctccgcgctctgctcacctctgacccggggcttcaccttggaactcctgggttttaggggcaaggaatgtcttacgttttcagtggtgctgctgcctgtgcacagttctgttcgcgtggctctgtgcaaagcacctgttctccatctctgggtagtggtaggagccggtgtggccccaggtgtccccactgtgcctgtgcactggccgtgggacgtcatggaggccatcccagggcagcaggggcatggggtaaagagatgtttatggggagtcttagcagaggaggctgggaaggtgtctgaacagtagatgggagatcagatgcccggaggatttggggtctcagcaaagagggccgaggtgggtgcaggtgagggtcgctggccccacccccgggaaggtgcagcagagctgtggctccccacacagcccggccagcacctgtgctctgggcatggctgtgctcctggaacgttccctgtcctggctggtcagggggtgcccctgccaagaatcgacaactttatcacagagggaagggccaatctgtggaggccacagggccagcttctgcctggagtcagggcaggtggtggcacaagcctcggggctgtaccaaagggcagtcgggcaccacaggcccgggcctccacctcaacaggcctcccgagccactgggagctgaatgccaggaggccgaagccctcgccccatgagggctgagaaggagtgtgagcatttgtgttacccagggccgaggctgcgcgaattaccgtgcacacttgatgtgaaatgaggtcgtcgtctatcgtggaaacccagcaagggctcacgggagagttttccattacaaggtcgtaccatgaaaatggtttttaacccgagtgcttgcgccttcatgctctggcagggagggcagagccacagctgcatgttaccgcctttgcaccagctccagaggcttgggaccaggctgtctcagttccagggtgcgtccggctcagaccgccctcctctctgccttctctctctgcctcaaatcttccctcgtttgcatctccctgacgcgtgcctgggccctcgtgcaagctgcttgactcctttccggaaacccttggggtgtgctggatacaggtgccactgaggactggaggtgtctgacactgtggttgaccccagggtccagctggcgtgcttggggcctccttgggccatgatgaggtcagaggagttttcccaggtgaaaactcctgggaaactcccagggccatgtgacctgccacctgctcctcccatattcagctcagtcttgtcctcatttccccaccagggtctctagctccgaggagctcccgtagagggcctgggctcagggcagggcggctgagtttccccacccatgtggggacccttgggtagtcgcttgattgggtagccctgaggaggccgagatgcgatgggccacgggccgtttccaaacacagagtcaggcacgtggaaggcccaggaatccccttccctcgaggcaggagtgggagaacggagagctgggccccgatttcacggcagccaggctgcagtgggcgaggctgtggtggtccacgtggcgctgggggcggggtctgattcaaatccgctggggctcggccttcctggcccgtgctggccgcgcctccacacgggcttggggtggacgccccgacctctagcaggtggctatttctccctttggaagagagcccctcacccatgctaggtgtttccctcctgggtcaggagcgtggccgtgtggcaaccccgggaccttaggcttatttatttgtttaaaaacattctgggcctggcttccgttgttgctaaatggggaaaagacatcccacctcagcagagttactgagaggctgaaaccggggtgctggcttgactggtgtgatctcaggtcattccagaagtggctcaggaagtcagtgagaccaggtacatggggggctcaggcagtgggtgagatgaggtacacggggggctcaggcagtgggtgaggccaggtacatggggggctcaggcactgggtgagatgaggtacacggggggctcaggcagagggtcagaccaggtacacgggggctctgatcacacgcacatatgagcacatgtgcacatgtgctgtttcatggtagccaggtctgtgcacacctgccccaaagtcccaggaagctgagaggccaaagatggaggctgacagggctggcgcggtggctcacacctgtagtcccagcactttgggaggccgaggcgagaggatcccttgagcccaggagtttaagaccagcctgagcaacatagtagaaccccatctctatgaaaaataaaaacaaaaattagctgagcatggtggtgtgcgcctgtagttccaatacttgggaggctgaagtgggaggatcacttgagcccaggaggtggaagctgcagtgagctgagattgcaccactgtactgcagcctgggtgacagagtgagagcccatctcaacaacaacaaagaagactgacaaatgcagtttcttggaaagaaacatttagtaggaacttaacctacacacagaagccaagtcggtgtctcggtgtcagtgagatgagatgatgggtcctcacaccatcaccccagacccagggtttatgcaccacaggggcgggtggctcagaagggatgcgcaggacgttgatatacgatgacatcaaggttgtctgacgaagggcaggattcatgataagtacctgctggtacacaaggaacaatggataaactggaaaccttagaggccttcccggaacaggggctaatcagaagccagcatggggggctggcatccaggatggagctgcttcagcctccacatgcgtgttcatacagatggtgcacagaaacgcagtgtacctgtgcacacacagacacgcagctactcgcacacacaagcacacacacagacatgcatgcatgcatccgtgtgtgtgcacctgtgcccatgaggaaacccatgcatgtgcattcatgcacgcacacaggcaccggtgggcccatgcccacacccacgagcaccgtctgattaggaggcctttcctctgacgctgtccgccatcctctcaggtttcacgcatgtgtgctgcagctcccatttcatcagcaagtttggaagaaccccacatttttcctgcgcgtcatctctgacacggcctccctctgctactccatcctgaaagccaagaacgcaggtatgtgcaggtgcctggcctcagtggcagcagtgcctgcctgctggtgttagtgtgtcaggagactgagtgaatctgggcttaggaagttcttaccccttttcgcatcaggaagtggtttaacccaaccactgtcaggctcgtctgcccgccctctcgtggggtgagcagagcacctgatggaagggacaggagctgtctgggagctgccatccttcccaccttgctctgcctggggaagcgctggggggcctggtctctcctgtttgccccatggtgggatttggggggcctggcctctcctgtttgccctgtggtgggattgggctgtctcccgtccatggcacttagggcccttgtgcaaacccaggccaagggcttaggaggaggccaggcccaggctaccccacccctctcaggagcagaggccgcgtatcaccacgacagagccccgcgccgtcctctgcttcccagtcaccgtcctctgcccctggacactttgtccagcatcagggaggtttctgatccgtctgaaattcaagccatgtcgaacctgcggtcctgagcttaacagcttctactttctgttctttctgtgttgtggaaatttcacctggagaagccgaagaaaacatttctgtcgtgactcctgcggtgcttgggtcgggacagccagagatggagccaccccgcagaccgtcgggtgtgggcagctttccggtgtctcctgggaggggagctgggctgggcctgtgactcctcagcctctgttttcccccagggatgtcgctgggggccaagggcgccgccggccctctgccctccgaggccgtgcagtggctgtgccaccaagcattcctgctcaagctgactcgacaccgtgtcacctacgtgccactcctggggtcactcaggacaggcaagtgtgggtggaggccagtgcgggccccacctgcccaggggtcatccttgaacgccctgtgtggggcgagcagcctcagatgctgctgaagtgcagacgcccccgggcctgaccctgggggcctggagccacgctggcagccctatgtgattaaacgctggtgtccccaggccacggagcctggcagggtccacaacttcttgaacccctgcttcccatctcaggggcgatggctccccacgcttgggagccttctgacccctgacctgtgtcctctcacagcctcttccctggctgctgccctgagctcctggggtcctgagcaagttctctccccgccccgccgctccagcgtcactgggctgcctgtctgctcgccccggtggaggggtgtctgtcccttcactgaggttcccaccagccagggccacgaggtgcaggccctgcctgcccggccacccacacgtcctaggagggttggaggatgccacctctggcctcttctggaacggagtctgattttggccccgcagcccagacgcagctgagtcggaagctcccggggacgacgctgactgccctggaggccgcagccaacccggcactgccctcagacttcaagaccatcctggactgatggccacccgcccacagccaggccgagagcagacaccagcagccctgtcacgccgggctctacgtcccagggagggaggggcggcccacacccaggcccgcaccgctgggagtctgaggcctgagtgagtgtttggccgaggcctgcatgtccggctgaaggctgagtgtccggctgaggcctgagcgagtgtccagccaagggctgagtgtccagcacacctgccgtcttcacttccccacaggctggcgctcggctccaccccagggccagcttttcctcaccaggagcccggcttccactccccacataggaatagtccatccccagattcgccattgttcacccctcgccctgccctcctttgccttccacccccaccatccaggtggagaccctgagaaggaccctgggagctctgggaatttggagtgaccaaaggtgtgccctgtacacaggcgaggaccctgcacctggatgggggtccctgtgggtcaaattggggggaggtgctgtgggagtaaaatactgaatatatgagtttttcagttttgaaaaaaa

(19) United States
(12) Patent Application Publication
(10) Pub. No.: US 2026/0092294 A1
(43) Pub. Date: Apr. 2, 2026

(54) METHODS OF IMMORTALIZING PRIMARY RESTING T CELLS BY CRISPR/DCAS9-BASED EPIGENETIC MODIFIERS AND TRANSCRIPTIONAL ACTIVATORS

(71) Applicant: City University of Hong Kong, Hong Kong (HK)

(72) Inventors: Raymond Hiu-Wai LAM, Hong Kong (HK); Chung TIN, Hong Kong (HK); Siping HUANG, Hong Kong (HK); Cia Hin LAU, Hong Kong (HK)

(21) Appl. No.: 18/901,172

(22) Filed: Sep. 30, 2024

(51) Int. Cl. C12N 15/90; C12N 5/0783; C12N 9/22; C12N 15/11

(52) U.S. Cl. CPC C12N 15/907; C12N 5/0636; C12N 9/22; C12N 15/11; C12N 2310/20

(57) ABSTRACT

A method for extending the replicative lifespan of T cells by activating the TERT gene is provided. Particularly, the method involves the epigenetic modification of the TERT promoter using a CRISPR/dCas9-based epigenetic modifier, followed by the reactivation of the modified TERT promoter using a Cas9-mediated transcriptional activator.

### REFERENCE TO SEQUENCE DISCLOSURE

The sequence listing file under the file name “P2659US01_Sequence Listing.xml” submitted in ST.26 XML file format with a file size of 43 KB created on Sep. 27, 2024 and filed on Sep. 30, 2024 is incorporated herein by reference.

### FIELD OF THE INVENTION

The present invention generally relates to the fields of molecular cell biology. More specifically the present invention relates to methods of immortalizing primary resting T cells.

### BACKGROUND OF THE INVENTION

Most mammalian somatic cells exhibit a finite proliferative lifespan due to the gradual shortening of telomeres, a process largely attributed to the deficiency of telomerase reverse transcriptase (TERT). While natural telomere erosion-induced cellular senescence is essential for protecting against issues such as cancer and age-related tissue/organ disorders, the immortalization of cells presents a sustainable source for specific cell types with consistent genetic and phenotypic characteristics, crucial for cell research and related medical applications. In the absence of telomerase, telomeres undergo shortening during cell replications, restricting proliferative capacity and culminating in permanent cell cycle arrest, known as replicative senescence (Hayflick and Moorhead, 1961). Conversely, the ectopic overexpression of the TERT transgene has proven to be an effective strategy for cell immortalization, successfully inhibiting replicative senescence (Barsov, 2011a; Hooijberg et al., 2000) and extending lifespan in various animal models (Mojiri et al., 2021; Tomas-Loba et al., 2008).

Given that current approaches for endogenous telomerase reactivation are both inefficient and short-lived, the TERT transgene can be incorporated into the genome of T cells using retrovirus or lentivirus vectors to achieve prolonged hTERT expression. Notably, the proliferative lifespan of T cells has been significantly expanded through the ectopic expression of the TERT transgene (Barsov, 2011a; b; Hooijberg et al., 2000). Despite the continuous cultivation of these immortalized T cells for over a year without any loss of primary cell functions and avoiding malignant transformation (Barsov, 2009; Barsov et al., 2006; Hooijberg et al., 2000), it's important to acknowledge that this viral-based transgenesis approach might introduce random insertional mutagenesis to the genome. Furthermore, prolonged drug selection for generating the desired genetically engineered cells may potentially alter cell phenotypes (Ranzani et al., 2013).

In recent years, there has been a remarkable advancement in biotechnology with the discovery of CRISPR/dCas9-based epigenetic modifiers and transcriptional activators. These tools enable the site-specific manipulation of gene expression without the necessity of altering the underlying DNA sequence (Hilton et al., 2015; Kearns et al., 2015).

Several CRISPR-based strategies have been reported for maintaining alternative splicing similar to unmodified cells. However, the utilization of CRISPR technology for immortalizing primary cells through the activation of an endogenous TERT gene has not been reported thus far. For instance, a CRISPR-based tool can generate various forms of TERT mRNA through post-transcriptional regulation (Patrick and Weng, 2019). This method overcomes some critical drawbacks associated with the ectopic overexpression approach, where the cloning expressions may miss some physiologically relevant splice variants if not specified by the cDNA cassette (La Russa and Qi, 2015). Nevertheless, challenges persist in this approach, including the potential oversight of certain physiologically relevant splice variants due to the overexpression of a specific splice variant from the open reading frame (ORF). Additionally, protein overexpression can be detrimental to the cell as it may disrupt the balance among protein complexes and the underlying cellular physiology.

Significantly, the underlying principle of CRISPR/dCas9-based tools underscores their suitability for telomerase-based cell immortalization, extending to primary monocytes and addressing the aforementioned challenges. Notably, epigenetic modifiers such as dCas9-p300 (Hilton et al., 2015) and dCas9-TET1 (Nunez et al., 2021) have been reported to activate target genes by inducing H3K27ac histone acetylation and DNA demethylation, respectively, at gene regulatory elements. This results in a direct alteration of chromatin states or epigenetic marks on promoter and enhancer regions, ensuring stable activation of target genes. Conversely, the transcriptional activators dCas9-VPR (Chavez et al., 2015) and dCas9-VPH (Tian et al., 2021) involve tripartite and fusion structures, respectively. Both are recognized as highly potent programmable transcriptional activators capable of transiently activating endogenous gene expressions by recruiting multiple components of the transcription pre-initiation complex or histone acetyltransferases to gene regulatory regions. This application of CRISPR/dCas9-based tools presents a promising avenue for precise and controlled gene expression modulation, especially in the context of cell immortalization.

Therefore, the present invention addresses the need of extending replicative lifespan of T cells by activating its endogenous TERT expression.

### SUMMARY OF THE INVENTION

It is an objective of the present invention to provide methods, or kits to solve the aforementioned technical problems.

In accordance with a first aspect of the present invention, a method of extending the replicative lifespan of a T cell by activating the TERT gene is provided. The method includes epigenetically modifying a TERT promoter of a T cell using a CRISPR/dCas9-based epigenetic modifier; and reactivating the modified TERT promoter using a Cas9-mediated transcriptional activator to obtain an immortalized T cell.

In accordance with one embodiment of the present invention, the epigenetically modifying includes a DNA demethylation and a histone modification.

In accordance with one embodiment of the present invention, the DNA demethylation results in a 10%-90% decrease in DNA methylation at the TERT promoter.

In accordance with one embodiment of the present invention, the histone modification includes a lysine residue acetylation of a histone protein.

In accordance with one embodiment of the present invention, the histone modification is an acetylation of the lysine residue at N-terminal position 27 of the histone H3 protein.

In accordance with one embodiment of the present invention, the CRISPR/dCas9-based epigenetic modifier comprises dCas9-p300 and dCas9-TET1.

In accordance with one embodiment of the present invention, the Cas9-mediated transcriptional activator induces target gene activation by recruiting multiple components of the transcription pre-initiation complex or histone acetyltransferases to the gene regulatory regions.

In accordance with one embodiment of the present invention, the Cas9-mediated transcriptional activator includes dCas9-VPR and dCas9-VPH.

In accordance with one embodiment of the present invention, the T cell is derived from peripheral blood mononuclear cells (PBMCs).

In accordance with one embodiment of the present invention, the reactivated TERT gene expression persists for a period of at least three months.

In accordance with one embodiment of the present invention, the immortalized T cell retains its primary T-cell characteristics following the replicative lifespan extension.

In accordance with one embodiment of the present invention, the method further includes assessing the viability of the immortalized T cell post-reactivation and confirming the absence of accelerated cell division.

In accordance with a second aspect of the present invention, a kit for extending a replicative lifespan of a T cell to obtain an immortalized T cell is provided. Particularly, the kit includes a CRISPR/dCas9-based epigenetic modifier and a Cas9-mediated transcriptional activator.

In accordance with one embodiment of the present invention, the CRISPR/dCas9-based epigenetic modifier includes dCas9-p300 and dCas9-TET1.

In accordance with another embodiment of the present invention, the Cas9-mediated transcriptional activator includes dCas9-VPR and dCas9-VPH.

In accordance with one embodiment of the present invention, the components are provided in a ready-to-use format, pre-packaged for direct application to T cells.

### BRIEF DESCRIPTION OF THE DRAWINGS

Embodiments of the invention are described in more details hereinafter with reference to the drawings, in which:

FIGS. 1A-1B depict the CRISPR constructs for targeted gene activation, in which FIG. 1A shows the targeted activation of endogenous human TERT by CRISPR/dCas9-based epigenetic modifiers and transcriptional activators with arrows indicating the sense or antisense orientation of sgRNAs (1, 2 and 3) designed to recognize target DNA sequences on the promoter, and FIG. 1B depicts the fluorescence microscopic images of GFP-transfected cells;

FIG. 2 depicts the TERT mRNA expression levels in PBMCs, THP-1-derived macrophages, and HEK293FT cells;

FIG. 3 depicts hTERT protein expression levels in PBMCs, THP-1-derived macrophages, and HEK293FT cells with the percentage of cells with the protein expression beyond the threshold level indicated in each plot;

FIGS. 4A-4C depict the delayed cellular senescence of resting T cells in PBMCs by activating endogenous hTERT, in which FIG. 4A shows the brightfield micrographs of cells were taken after two months of nucleofection, FIG. 4B displays the cell coverage of the CRISPR-treated (dCas9-p300, dCas9-TET1, dCas9-VPH, and dCas9-VPR) PBMCs as a function of time where the cell coverage is normalized such that it initially equals ‘1’, and FIG. 4C demonstrates the forward scatter parameter (FSC) of the flow cytometry measurement for the untreated PBMCs before nucleofection (untreated) and the transfected PBMCs after cultured for two months (transfected); and

FIGS. 5A-5C depict the characterizations of engineered-T cells, in which FIG. 5A displays the PI-based cell cycle analysis of CRISPR/dCas9-based immortalized T cells, Jurkat T cells, and PBMC cells, FIG. 5B shows the flow cytometry analysis of engineered-T cells, Jurkat-T cells, and PBMC cells upon labelling with CFDA-SE (0 hour) and after culturing of these labeled cells for 72 hours, and FIG. 5C shows the annexin V and PI-labeled cells in the engineered-T cell population that have been cultured for 3 months before cell viability was carried out.

### DETAILED DESCRIPTION

In the following description, methods and/or kits of extending a replicative lifespan of a T cell by activating the TERT gene and the likes are set forth as preferred examples. It will be apparent to those skilled in the art that modifications, including additions and/or substitutions may be made without departing from the scope and spirit of the invention. Specific details may be omitted so as not to obscure the invention; however, the disclosure is written to enable one skilled in the art to practice the teachings herein without undue experimentation.

In accordance with a first aspect of the present invention, a method for extending the replicative lifespan of a T cell by activating the TERT gene is provided. Particularly, the method involves a series of steps. Firstly, the TERT promoter of a T cell is epigenetically modified using a CRISPR/dCas9-based epigenetic modifier. This modification includes both DNA demethylation and histone modification. The DNA demethylation step results in a 10%-90% decrease in DNA methylation at the TERT promoter. Concurrently, a lysine residue acetylation of a histone protein, specifically at N-terminal position 27 of the histone H3 protein, constitutes the histone modification. The CRISPR/dCas9-based epigenetic modifier employed in this method includes dCas9-p300 and dCas9-TET1.

Following the epigenetic modification, the modified TERT promoter is reactivated using a Cas9-mediated transcriptional activator. This transcriptional activator induces target gene activation by recruiting multiple components of the transcription pre-initiation complex or histone acetyltransferases to the gene regulatory regions. In this context, the Cas9-mediated transcriptional activator includes dCas9-VPR and dCas9-VPH.

The T cell subjected to this method is derived from peripheral blood mononuclear cells (PBMCs). Notably, the reactivated TERT gene expression persists for a substantial period, specifically for at least three months. The resulting immortalized T cell retains its primary T-cell characteristics even after the extension of its replicative lifespan. The viability of the immortalized T cell post-reactivation is assessed, and it is confirmed that there is no accelerated cell division, ensuring the maintenance of its intended characteristics. This method provides a robust and sustainable approach to extending the replicative lifespan of T cells, promising longevity and functional preservation.

In accordance with a second aspect of the present invention, a kit for extending the replicative lifespan of a T cell to obtain an immortalized T cell is provided. The kit includes a CRISPR/dCas9-based epigenetic modifier and a Cas9-mediated transcriptional activator. Specifically, the CRISPR/dCas9-based epigenetic modifier included in the kit consists of dCas9-p300 and dCas9-TET1. These components are designed to effectively induce DNA demethylation and histone modification at the TERT promoter, crucial steps for achieving robust and sustained TERT gene activation.

Moreover, the Cas9-mediated transcriptional activator present in the kit includes dCas9-VPR and dCas9-VPH. This transcriptional activator is adept at recruiting multiple components of the transcription pre-initiation complex or histone acetyltransferases to the gene regulatory regions, ensuring the comprehensive reactivation of the modified TERT promoter.

An additional feature of the kit is its user-friendly design. All components are provided in a ready-to-use format, pre-packaged for direct application to T cells. This facilitates the convenient and efficient application of the kit, streamlining the process of extending the replicative lifespan of T cells and obtaining immortalized T cells. Overall, the kit represents a comprehensive and user-friendly solution for researchers and practitioners aiming to employ the disclosed method for T cell immortalization.

Traditional approaches for primary cell immortalization involve introducing exogenous telomerase, achieved by overexpressing the TERT transgene in an episomal form or integrating it into the genome using retrovirus or lentivirus vector transduction. The present invention provides a different approach, demonstrating the efficacy of CRISPR/dCas9-based epigenetic modifiers (p300 histone acetyltransferase and TET1 DNA demethylase) and transcriptional activators (VPH and VPR) in reactivating the endogenous TERT gene. This method addresses limitations associated with exogenous telomerase-related techniques for primary cell immortalization. It showcases a combined use of these epigenetic modifiers and transcriptional activators, resulting in robust and enduring endogenous TERT reactivation in primary cells. Importantly, the research successfully extended the lifespan of resting T cells, delaying cellular senescence for at least three months without impacting the expression of a crucial T-cell marker (CD3) or inducing accelerated cell division. This strategy for cell immortalization holds promise for broader application across various primary human cell types.

In the present invention, the replicative lifespan of T cells derived from primary peripheral blood mononuclear cells (PBMCs) is successfully prolonged by activating their endogenous TERT expression through the utilization of CRISPR/dCas9-based epigenetic modifiers (dCas9-p300 and dCas9-TET1) and transcriptional activators (dCas9-VPH and dCas9-VPR). Furthermore, it is demonstrated that the combined use of these epigenetic modifiers and transcriptional activators results in more robust and enduring endogenous TERT expressions in primary cells. Importantly, these programmable CRISPR tools have the potential to immortalize various human/mammalian cells, addressing some of the limitations associated with exogenous telomerase-related methods.

A primary objective of the current invention is to generate immortalized cells derived from diverse primary cell types, specifically for applications in cell research and therapy. These epigenetically reprogrammed immortalized cells exhibit a closer resemblance to their native state compared to genetically modified cells.

Reactivating endogenous telomerase, as proposed in this invention, offers advantages over methods involving exogenous telomerase overexpression. It mitigates potential host immune responses, enhancing the safety of adoptive transfer of TERT-expressing T cells in vivo. Targeted reactivation of endogenous telomerase also minimizes the risk of genomic instability and malignant transformation in T cells, ensuring a physiological level of TERT expression.

### EXAMPLES

### Example 1. Targeted Activation of Endogenous TERT Promoter by Multiple Single Guide RNAs

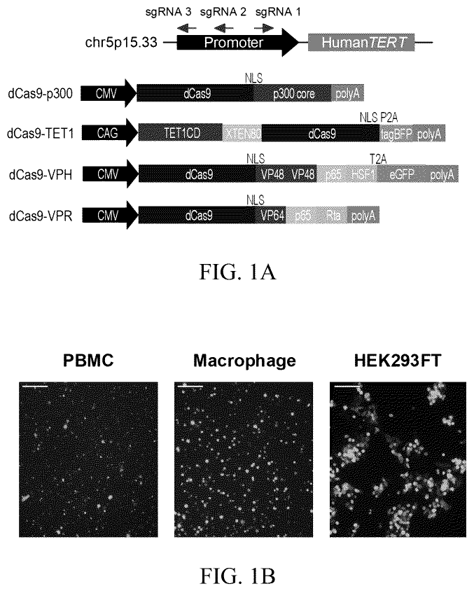

Utilizing CRISPR/dCas9-based epigenetic modifiers (dCas9-p300 and dCas9-TET1) and transcriptional activators (dCas9-VPH and dCas9-VPR), the endogenous human TERT gene (SEQ ID NO: 01) is activated by precise targeting of the TERT promoter. For synergistic activation, three sgRNAs are strategically designed to target the TERT promoter (FIG. 1A). These sgRNAs are positioned at −157 bp, −333 bp, and −499 bp from the transcription start site (TSS) of the TERT gene. Given that the human U6 promoter drives sgRNA expression, all sgRNAs feature a “G” at the 5′ end to initiate transcription. The 20 bp target sequence is immediately followed by a 3-bp 5′-NGG-3′ PAM sequence. Notably, all designed sgRNA target sequences exhibit no potential off-target sites in the human genome.

In the CRISPR guide sequence design process, dCas9-p300 (Addgene #61357), dCas9-TET1 (Addgene #167983), dCas9-VPH (Addgene #158091), and dCas9-VPR (Addgene #63798) are procured from Addgene (Watertown, MA, USA). For the construction of the required sgRNA guide sequence, a pair of annealed oligonucleotides is cloned into a pU6-sgRNA expression cassette (Addgene #53188) featuring the sgRNA scaffold backbone and tracrRNA, using BbsI. The oligonucleotides are designed based on the target site sequence (20 bp), flanked on the 3′ end by a 3-bp 5′-NGG-3′ PAM sequence. The CRISPR RGEN Tool, Cas-Designer, is employed to identify the target sequence of sgRNAs. To maximize the U6 promoter activity, the first nucleotide of the transcribed gRNA is a guanine nucleotide (G). The selected sgRNA target sequences have no potential off-target sites of RNA-guided endonucleases within 2-nt mismatches. DNA sequencing, utilizing the pU6-seq primer, is conducted to confirm the successful insertion of the guide sequence in the sgRNA.

### Example 2. Transfection Efficiency of PBMCs, THP1-Derived Macrophages and HEK293FT Cells

Human peripheral blood mononuclear cells (PBMCs; ATCC, NY, USA) are cultivated in complete RPMI-1640 culture medium supplemented with 10% fetal bovine serum (Gibco, NY, USA), and 100 IU/ml IL-2 (STEMCELL, Vancouver, Canada). PBMCs undergo a thawing process and are incubated for 2-3 days before nucleofection to ensure viability and proper cellular behaviors. THP-1 and Jurkat cells (ATCC, NY, USA) are sustained in RPMI-1640 medium containing 10% FBS (Thermo Fisher, NY, USA). THP-1 cells (2×10⁵/ml) are differentiated into MO macrophages using 200 nM phorbol 12-myristate 13-acetate (PMA, Sigma-Aldrich, Missouri, USA) for 2 days. HEK293FT cells are cultured in Dulbecco's modified Eagle's medium (DMEM) (high glucose) (Gibco, NY, USA) with 10% fetal bovine serum (Gibco, NY USA). All cells are maintained under standard cell culture conditions (37° C., 5% CO₂) in a humidified incubator (Thermo, NY, USA).

PBMCs encompass various peripheral blood cell types, including CD3+ T cells, B cells, NK cells, monocytes, and dendritic cells, with the proportions of these populations differing among individuals. Given the challenge of transfecting human leukocytes with widely used liposome-based transfection reagents like Lipofectamine 3000, nucleofection emerges as the preferred method for introducing plasmids into PBMCs, employing optimized settings. Nucleofector system (Lonza, Basel, Switzerland) is employed for nucleofection of the plasmid into both PBMCs and THP-1-derived macrophages. Plasmid DNAs (total weight: 1000 ng) are introduced to the cells, with an equal allocation for each plasmid vector, i.e., 500 ng of epigenetic modifiers (and transcriptional activators), and 500 ng of sgRNAs. The cell/DNA mixture is transferred into the system for nucleofection, after which the transfected cells are supplemented with prewarmed medium and subsequently transferred to a well in a 24-well plate.

To determine the optimal nucleofection conditions, various experimental parameters are systematically investigated using macrophages derived from the human monocytic cell line THP-1. For instance, when nucleofecting 1×10⁶ cells in 20 μl of reagents, a vector dose of 1000 ng is identified as yielding the highest transfection efficiency. Notably, while hTERT expression in THP-1 cells is well-documented, THP-1-derived macrophages exhibit a notably low hTERT expression. Hence, the investigation aims to explore whether the CRISPR/dCas9-based approach can effectively transfect THP-1-derived macrophages, inducing expressions of TERT mRNA and the corresponding hTERT protein. To assess transfection efficiency, pCMV-GFP plasmids are transfected into THP-1-derived macrophages and PBMCs using the optimized nucleofection procedures, counting cells with and without GFP expression (FIG. 1B). Results indicate successful transfection rates of 15.46% and 19.54% for THP-1-derived macrophages and PBMCs, respectively.

Conversely, HEK293FT, a highly transfectable clonal isolate derived from human embryonal kidney cells transformed with the SV40 large T antigen, enables exceptionally high expression levels of CRISPR components. The presence of SV40 large T antigen facilitates robust expression of recombinant proteins. Therefore, pCMV-GFP plasmids are transfected into HEK293FT cells using Lipofectamine 3000 with an efficiency of 69.35% (FIG. 1B). Briefly, one day prior to plasmid transfection, 1×10⁵ HEK293FT cells are seeded in one well (area: 1.9 cm²) of a 24-well plate. For transfection, 500 ng of CRISPR/dCas9-based epigenetic modifiers (and transcriptional activators) targeting the human TERT promoter are mixed with 1 μL of P3000 Reagent and 1.5 μL of Lipofectamine 3000 (Invitrogen, NY, USA) in 200 μL of Opti-MEM I Reduced Serum Medium (Gibco, NY, USA). The transfection mixture is added to the cell cultures and incubated overnight. On day ‘1’, the Opti-MEM medium is changed to DMEM medium with 10% FBS. On day ‘2’, some of the transfected cells are harvested for total RNA extraction and qPCR tests. On day ‘3’, the remaining cells are harvested for flow cytometry measurements of hTERT.

### Example 3. Targeted Upregulation of Endogenous TERT mRNAs and Proteins

The chosen cell types undergo the same procedures as described above for transfection with GFP and the CRISPR plasmids. Two plasmid mixtures are employed: 1) the epigenetic modifiers (dCas9-p300 and dCas9-TET1) and 2) both the epigenetic modifiers and the transcriptional activators (dCas9-VPH and dCas9-VPR).

To assess the efficacy of the designed sgRNAs and CRISPR tools, quantitative polymerase chain reaction (qPCR) is conducted to quantify hTERT mRNA. Total RNA is extracted from the cells using the RNeasy Mini Kit (QIAGEN, Hilden, Germany). The concentration and purity of the total RNAs are determined using a Biochrom spectrophotometer. After obtaining RNA with the required purity and quantity, reverse transcription is performed using the SuperScript III First-Strand Synthesis System (Invitrogen, NY, USA) following the manufacturer's protocol.

For qPCR, the SsoAdvanced Universal SYBR Green Supermix (Bio-Rad, California, USA) is used to amplify the synthesized cDNA. Each qPCR reaction mixture contains 2× SsoAdvanced Universal SYBR Green Supermix, forward primer, reverse primer, cDNA sample, and nuclease-free water. An exon-exon junction primer is designed to avoid genomic DNA amplification. The qPCR is carried out using a Thermal Cycling (Bio-Rad, California, USA) Connect System with the following procedures: an initial denaturation step at 95° C. for 3 min followed by 40 cycles at 95° C. for 15 s and 62° C. for 45 s. The housekeeping gene glyceraldehyde 3-phosphate dehydrogenase (GAPDH) is used for internal normalization. Relative gene expression is calculated using the 2-ΔΔCt method. Technical triplicates for each sample are performed. A melt curve analysis is conducted at 65° C.-95° C. with 0.5° C. increments and 2-5 s/step to verify primer specificity and ensure no primer-dimer formation during qPCR.

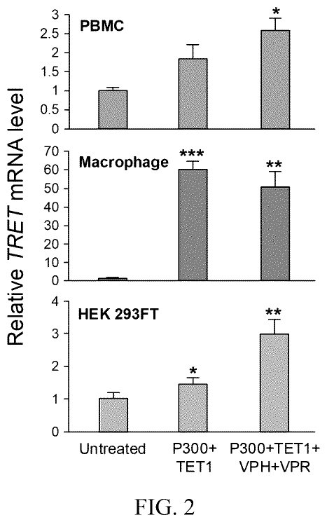

In FIG. 2, the outcomes indicate that co-transfection with the epigenetic modifiers results in an upregulation of hTERT mRNA expressions in PBMCs (1.8 times), THP-1-derived macrophages (61.2 times), and HEK293FT cells (1.4 times). However, the modest upregulation in HEK293FT suggests that the epigenetic modifiers alone might not be adequate to induce target gene activation across all cell types. Furthermore, the results demonstrate that co-transfection with both the epigenetic modifiers and the transcriptional activators induces a substantial upregulation of hTERT mRNA in all selected cell types: PBMCs (2.6 times), THP-1-derived macrophages (50.5 times), and HEK293FT cells (2.9 times).

To assess the impact of the current sgRNAs and CRISPR tools on protein expressions, flow cytometry is conducted. In brief, a flow cytometer (BD Biosciences, New Jersey, USA) is employed to measure CD3 and hTERT expressions in cells. For CD3 expression in PBMCs, cells are stained with FITC-conjugated anti-human CD3 for 30 min at 4° C. in the dark, followed by two washes with PBS. To determine hTERT protein expression levels, cells are initially fixed in 4% PFA (Thermo Fisher, NY, USA) for 20 min at 4° C. After two washes with PBS, the cells are permeabilized in 0.1% Triton X-100 (Sigma, Missouri, USA) for 30 min. After two washes with PBS, the cells are stained with FITC-conjugated anti-human hTERT (Santa Cruz, Dallas, Texas, USA) for 1 h at 4° C., followed by washing and resuspending with 1×10⁵ cells in PBS for flow cytometry analysis. FITC signals of the stained cells are then quantified by the flow cytometer. Additionally, forward scatter (FSC) and side scatter (SSC) signals are used to quantify cell size and granularity, respectively.

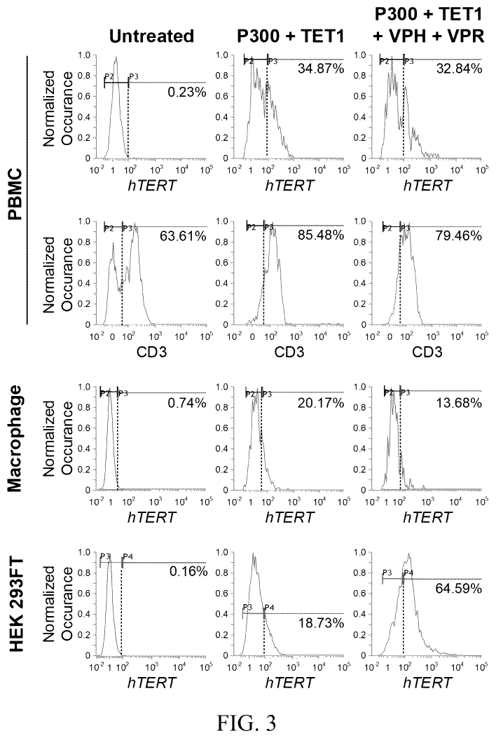

In FIG. 3, the quantification of TERT protein in the selected transfected cells is depicted. Measurements are conducted four days post-plasmid transfection using flow cytometry. FITC-labeled anti-TERT antibodies are employed to stain the cells before assessment. The lower panels for PBMCs indicate CD3 expression, a T-cell marker, in PBMCs stained with FITC-labeled anti-CD3 antibodies, measured two months post-nucleofection via flow cytometry. The occurrence (count over the total cell number) for each case is scaled such that the maximum value equals 1. A threshold level (vertical hidden line) for each cell type is established such that >99% of the corresponding unstained and untreated cells fall below the threshold level of protein expression. The percentage of cells with protein expression beyond the threshold level is indicated in each plot.

As anticipated, both the epigenetic modifiers and the transcriptional activators lead to an upregulation of TERT expression. Additionally, examination of CD3 expression in PBMCs two months after the transfection process reveals an enrichment of CD3 upon telomerase reactivation in treated PBMCs, indicating the survival and successful transfection of the T cells. Collectively, these findings illustrate that the CRISPR/dCas9-based approach effectively activates endogenous TERT expression in resting primary T cells. This approach likely induces delayed cellular senescence of resting T cells for at least two months without compromising key immunoactivities, as evidenced by the prolonged CD3 expression.

### Example 4. Proliferation of the TERT-Activated Resting T Cells

The cell density of PBMCs upon nucleofection are cultured and monitored up to two months. Cell proliferation assays are carried out with CFSE stains (CFDA-SE (5(6)-carboxyfluorescein diacetate succinimidyl ester, Thermo Fisher). Briefly, the initial cell density is 1×10⁶ cells/ml. Fluorescence intensity levels in cells 1) immediately after CFSE staining (Iₒ) and 2) 72-hour after staining (In) are measured to determine the proliferation rate. Because cell division can be visualized as a series of generational divisions that each result in halving the fluorescence intensity, the number of generations (n) can be estimated by solving Iₒ=2ⁿIₙ. Additionally, to discriminate viability of the immortalized T cells, annexin V (Thermo Fisher) is used to detect phosphatidylserine expression over apoptotic cells and PI stains is used to stain intracellular DNAs according to its capability of penetrating via membranes of late apoptotic/dead cells. Cells are resuspended with PBS before staining with annexin V and PI for 30 min at room temperature. The stained cells are washed with PBS before analyzed with flow cytometry.

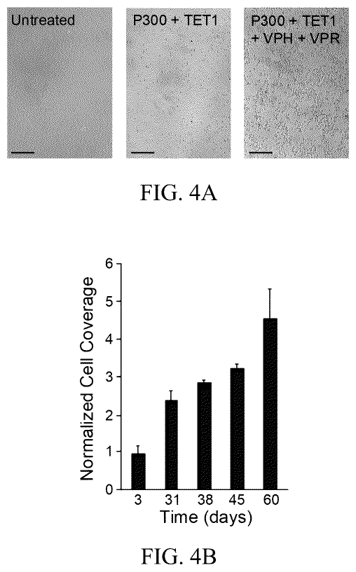

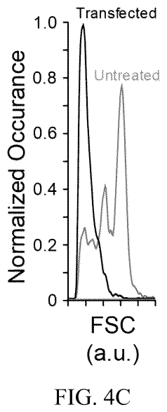

As shown in FIG. 4A, PBMCs are transfected with a combination of dCas9-p300 and dCas9-TET1 or a combination of four different CRISPR plasmids (dCas9-p300, dCas9-TET1, dCas9-VPH, and dCas9-VPR) and the initial cell density among treatment groups is the same (1×10⁶ cells/ml). The results show that the majority of the untreated PBMCs die after being cultured for about two months, whereas TERT-activated PBMCs remain alive and continue to proliferate. The cells transfected with epigenetic modifiers and transcriptional activators exhibit a higher proliferation rate than the cells transfected with only epigenetic modifiers, implying a more robust endogenous TERT activation in the former case. The population expansion of PBMCs transfected with both the epigenetic modifiers and the transcriptional activators are also monitored. As shown in FIG. 4B, the brightfield microscopes is taken to quantify the cell coverage using image processing software (Image J, NIH) at different time points of the cell culture. The cell density reaches a sufficient level for subculture after incubation for two months. Furthermore, the distribution of cell size in the untreated PBMCs and the expanded and transfected PBMCs are examined, stained and measured by the forward scatter parameter (FSC) in flow cytometry as shown in FIG. 4C. The untreated cells contain three dominant size ranges. The largest group belongs to monocytes and B-cells, the medium-sized belongs to NK cells and some granulocytes, and the smallest group belongs to T cells and possibly a small portion of dendritic cells. Hence, this result suggests enrichment of CD3+ T cells population upon the nucleofection of PBMCs, agreeing with the positive CD3 expression as shown in FIG. 3.

### Example 5. Characterization of Immortalized T Cells

The cell cycle and proliferation rate of the CRISPR/dCas9-based immortalized T cells are further characterized and compared with those of Jurkat cells, which are a widely used immortalized human T-cell line for studying T cell leukaemia, and with those of PBMCs where the T-cell is derived from. PI stain is used to determine the four phases of cell cycle, i.e., cell resting/enlargement (G1), DNA synthesis(S), preparation for cell division (G2), and splitting (M). Further, cell cycles of Jurkat, immortalized T cells and PBMC cells are determined through the measurement of DNA contents in each cell using flow cytometry. Briefly, cells are firstly fixed with 70% cold alcohol, followed by treating them with ribonuclease A (50 μl of 100 μg/ml; Sigma) to remove RNAs from the cell (PI; 200 μl from a 50 μg/ml stock solution purchased from Thermo Fisher). Distributions of cells in the G1, S, and G2 cell cycles are quantified according to the stoichiometric of PI stains.

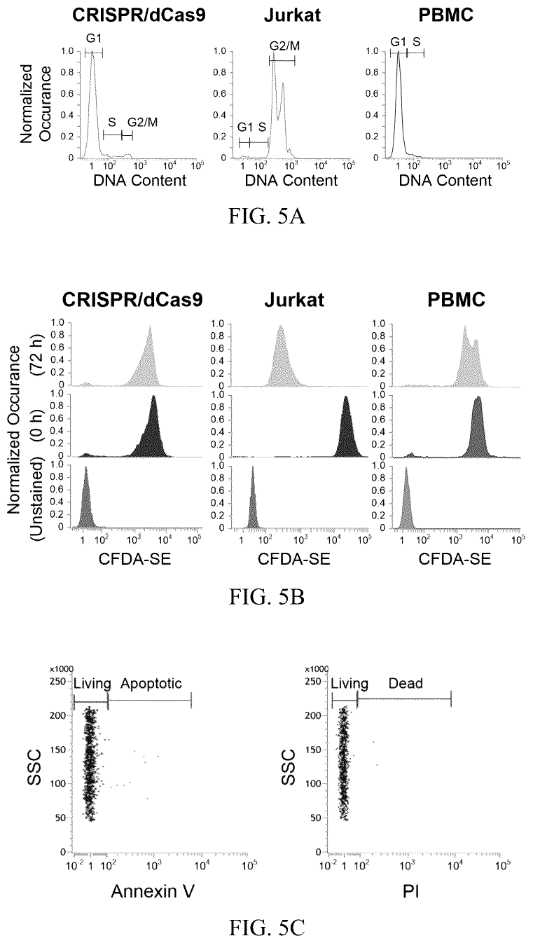

As shown in FIG. 5A, the results reveal that the majority of the CRISPR/dCas9-based immortalized T cells and PBMCs is in the resting state (G1), while the majority of Jurkat cells is in the dividing states (G2/M), implying that the lifespan of the CRISPR/dCas9-based immortalized T cells is successfully extended without significantly accelerated cell division, which can be an indicator of tumorigenicity.

In addition, CFDA-SE is used to label the CRISPR/dCas9-based immortalized T cells and control cells, which are subsequently cultured for 72 h. In principle, cell division of a cell results in redistributing half of its fluorescence in each of the two divided cells in the next generation, implying that reduction of the fluorescence can reflect the cell division. The fluorescence intensity of the immortalized T cells, Jurkat cells and PBMCs before and after CFDA-SE staining for 72 hours is shown in FIG. 5B. The proliferation rate of the immortalized cells (about one division over 72 hours) is similar to PBMCs, but is much lower than the Jurkat cells (about nine divisions over 72 hours), implying that the immortalized T cells fail to show an overaccelerated cell division rate as seem in the cancerous cells grows. Further, annexin V staining is applied for detecting apoptotic cells and PI staining for identifying dead cells in CRISPR/dCas9-based immortalized cells that have been cultured over three months. As shown in FIG. 5C, the results support that the immortalized T cells remained viable where only 2.47% of the cultured cells are apoptotic, indicated by the higher annexin V expressions and 0.41% of the cells are late apoptotic/dead cells, indicated by the PI staining.

In summary, CRISPR/dCas9-based epigenetic modifiers and transcriptional activators that the present invention utilized to activate the endogenous TERT promoter do not alter the DNA sequence at this genomic locus or induce any DNA damage to the genome. This method triggers physiological or near-physiological expression of endogenous TERT gene by directly modulating its regulatory elements. In other words, it specifics downstream expressions of the most relevant splice variants from the TERT gene.

It is shown that the life span of unstimulated T lymphocytes has been successfully extended for at least three months without malignant transformation or loss of primary cell functions. These epigenetic tools may be used to immortalize the other cell types while maintaining primary cell characteristics.

The present invention showcases the presence of immortalized T-cells in transfected PBMCs, substantiated through the sustained CD3 expression, consistent cell size distribution, and ongoing cell proliferation. Beyond PBMC-derived T cells, the efficacy of these CRISPR tools is confirmed in HEK293FT and THP-1-derived macrophages. Notably, CRISPR/dCas9-based epigenetic modifiers and transcriptional activators robustly enhance TERT expression in HEK293FT and THP-1-derived macrophages, even in the absence of detectable hTERT expression at a baseline level. It is anticipated that the outlined strategy for cell immortalization can be broadly applied to diverse primary human cells.

The foregoing description of the present invention has been provided for the purposes of illustration and description. It is not intended to be exhaustive or to limit the invention to the precise forms disclosed. Many modifications and variations will be apparent to the practitioner skilled in the art.

The embodiments were chosen and described in order to best explain the principles of the invention and its practical application, thereby enabling others skilled in the art to understand the invention for various embodiments and with various modifications that are suited to the particular use contemplated.

1. A method of extending the replicative lifespan of a T cell by activating the TERT gene, comprising:
epigenetically modifying a TERT promoter of a T cell using a CRISPR/dCas9-based epigenetic modifier; and
reactivating the modified TERT promoter using a Cas9-mediated transcriptional activator to obtain an immortalized T cell.

2. The method of claim 1, wherein the epigenetically modifying comprises a DNA demethylation and a histone modification.

3. The method of claim 2, wherein the DNA demethylation results in a 10%-90% decrease in DNA methylation at the TERT promoter.

4. The method of claim 2, wherein the histone modification comprises a lysine residue acetylation of a histone protein.

5. The method of claim 4, wherein the histone modification is an acetylation of the lysine residue at N-terminal position 27 of the histone H3 protein.

6. The method of claim 1, wherein the CRISPR/dCas9-based epigenetic modifier comprises dCas9-p300 and dCas9-TET1.

7. The method of claim 1, wherein the Cas9-mediated transcriptional activator induces target gene activation by recruiting multiple components of a transcription pre-initiation complex or histone acetyltransferases to gene regulatory regions.

8. The method of claim 7, wherein the Cas9-mediated transcriptional activator comprises dCas9-VPR and dCas9-VPH.

9. The method of claim 1, wherein the T cell is derived from peripheral blood mononuclear cells (PBMCs).

10. The method of claim 1, wherein the reactivated TERT gene expression persists for a period of at least three months.

11. The method of claim 1, wherein the immortalized T cell retains its primary T-cell characteristics following the replicative lifespan extension.

12. The method of claim 1, further comprising assessing the viability of the immortalized T cell post-reactivation and confirming the absence of accelerated cell division.

13. A kit for extending a replicative lifespan of a T cell to obtain an immortalized T cell, comprising:
a CRISPR/dCas9-based epigenetic modifier; and
a Cas9-mediated transcriptional activator.

14. The kit of claim 13, wherein the CRISPR/dCas9-based epigenetic modifier comprises dCas9-p300 and dCas9-TET1.

15. The kit of claim 13, wherein the Cas9-mediated transcriptional activator comprises dCas9-VPR and dCas9-VPH.

16. The kit of claim 13, wherein the components are provided in a ready-to-use format, pre-packaged for direct application to T cells.
